(19) United States
(12) Patent Application Publication
(10) Pub. No.: US 2026/0284154 A1
(43) Pub. Date: Sep. 24, 2026

(54) COMPOSITION AND METHODS FOR MODULATION OF ELOVL2

(71) Applicants: YOUHEALTH BIOTECH, LIMITED, Grand Cayman (KY); THE REGENTS OF THE UNIVERSITY OF CALIFORNIA, Oakland, CA (US)

(72) Inventors: Kang Zhang, Macau (MO); Xin Li, Shenyang (CN); Gen Li, Shenyang (CN)

(73) Assignees: YOUHEALTH BIOTECH, LIMITED, Grand Cayman (KY); THE REGENTS OF THE UNIVERSITY OF CALIFORNIA, Oakland, CA (US)

(21) Appl. No.: 19/179,951

(22) Filed: Apr. 15, 2025

Related U.S. Application Data
(63) Continuation of application No. 18/970,544, filed on Dec. 5, 2024, now abandoned.
Application No. 18/970,544 is a continuation of application No. 17/122,457, filed on Dec. 15, 2020, now abandoned.
Application No. 17/122,457 is a continuation of application No. PCT/US19/37344, filed on Jun. 14, 2019.
(60) Provisional application No. 62/685,768, filed on Jun. 15, 2018.

(51) Int. Cl. A61K 38/45; A61K 31/202; A61K 31/455; A61K 35/76; A61K 35/761

(52) U.S. Cl. CPC A61K 38/45; A61K 31/202; A61K 31/455; A61K 35/76; A61K 35/761; A61K 35/763; A61K 38/1709; A61K 48/0066; A61P 39/06

(57) ABSTRACT

Disclosed herein are therapeutic agents capable of increasing the expression level of an epigenetic marker described herein. Also described herein are therapeutic agents that reduce or slow-down an aging phenotype.

### CROSS-REFERENCE TO RELATED APPLICATIONS

This application is a continuation of U.S. application Ser. No. 18/970,544, filed Dec. 5, 2024, which is a continuation of U.S. application Ser. No. 17/122,457, filed Dec. 15, 2020, which is a continuation of International Patent Application No. PCT/US2019/037344, filed Jun. 14, 2019, which claims the benefit of U.S. Provisional Application No. 62/685,768, filed Jun. 15, 2018, the disclosures of which applications are herein incorporated by reference in their entirety for all purposes.

### REFERENCE TO AN ELECTRONIC SEQUENCE LISTING

The contents of the electronic sequence listing (YH1BIO0001-4 US_ST26.xml; Size: 8,227 bytes; and Date of Creation: Apr. 14, 2025) is herein incorporated by reference in its entirety.

### BACKGROUND OF THE DISCLOSURE

The rate and progression of aging varies from person to person and are further influenced by environmental factors, lifestyle choices, and/or physical fitness. In some instances, studies have shown that the state of the epigenome (e.g., mutation within the genome and/or methylation) correlate with age. As such, DNA methylation are utilized, for example, for determining age or changes in the rate of aging based on environmental factors, lifestyle choices, and/or physical fitness.

### SUMMARY OF THE DISCLOSURE

Provided herein are therapeutic agents capable of increasing the expression level of an epigenetic marker described herein. Also described herein are therapeutic agents that reduce or slow-down an aging phenotype.

Disclosed herein, in certain embodiments, is a method of treating a subject in need thereof, comprising: administering to the subject a composition comprising an active agent that up-regulates ELOVL fatty acid elongase 2 (ELOVL2) expression and a pharmaceutically acceptable carrier.

In some embodiments, the active agent comprises a vector comprising a polynucleotide encoding ELOVL2 or a functionally-active fragment thereof.

In some embodiments, the polynucleotide encodes a polypeptide comprising at least 80%, 85%, 90%, 95%, or 99% sequence identity to SEQ ID NO: 1.

In some embodiments, the vector comprises a viral vector. In some embodiments, the viral vector comprises an adeno-associated virus (AAV)-based vector. In some embodiments, the AAV-based vector comprises AAV-based vector of serotype 1 (AAV1), AAV-based vector of serotype 2 (AAV2), AAV-based vector of serotype 3 (AAV3), AAV-based vector of serotype 4 (AAV4), AAV-based vector of serotype 5 (AAV5), AAV-based vector of serotype 6 (AAV6), AAV-based vector of serotype 7 (AAV7), AAV-based vector of serotype 8 (AAV8), AAV-based vector of serotype 9 (AAV9), or a humanized AAV-based vector. In some embodiments, the viral vector comprises an adenovirus-based vector, an alphavirus-based vector, a herpesvirus-based vector, a retrovirus-based vector, a lentivirus-based vector, or a vaccinia virus-based vector.

In some embodiments, the vector comprises a cell or tissue-specific promoter. In some embodiments, the cell or tissue-specific promoter is an endogenous promotor specific to the cell type of interest. In some embodiments, the cell or tissue-specific promoter is an exogenous promotor specific to the cell type of interest.

In some embodiments, the vector comprises a microbial promoter. In some embodiments, the microbial promoter comprises SV40 or cytomegalovirus (CMV) immediate-early promoter.

In some embodiments, the vector comprises an enhancer, an inverted terminal repeats (ITR), a capsid, polyadenylation signal, a signal sequence, or a combination thereof.

In some embodiments, the vector comprises a selectable marker. In some embodiments, the selectable marker comprises a polynucleotide encoding a fluorescent protein. In some embodiments, the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyanl, Midori-Ishi Cyan, TagCFP, mTFP1, enhanced yellow fluorescent protein (EYFP), Topaz, MCitrine, YPet, TagYFP, PhiYFP, ZsYellowl, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRedl, mRaspberry, dKeima-Tandem, mPlum, or AQ143.

In some embodiments, the vector comprises a polynucleotide encoding an elongation factor 1-alpha (EF1α).

In some embodiments, the vector comprises a polynucleotide encoding a Klarsicht, ANC-1, Syne Homology (KASH) domain.

In some embodiments, the active agent inhibits activation of chromodomain-helicase-DNA-binding protein 4 (CHD4).

In some embodiments, the composition is administered systemically. In some embodiments, the composition is administered as a local injection. In some embodiments, the composition is formulated for parenteral administration. In some embodiments, the composition is formulated for oral or intranasal administration.

In some embodiments, a reduced ELOVL2 expression level correlates with an increase in accumulation of a plurality of fatty acids with less than 22 carbon chains. In some embodiments, the plurality of fatty acids comprises saturated fatty acids, monounsaturated fatty acids, or a combination thereof.

In some embodiments, an elevated expression of ELOVL2 reduces or slows-down an aging phenotype. In some embodiments, the aging phenotype comprises hair loss, a decrease in bone density, a decrease in endurance, a decrease in muscle strength, or neurodegeneration. In some embodiments, an elevated expression of ELOVL2 treats age-related macular degeneration (AMD).

In some embodiments, the subject is human.

Disclosed herein, in certain embodiments, is a method of reducing or slowing-down an aging phenotype in a subject in need thereof, comprising administering to the subject a composition comprising a therapeutic agent that reduces or slows-down the aging phenotype.

In some embodiments, the therapeutic agent comprises a C₁₈-C₂₈ polyunsaturated fatty acid. In some embodiments, the therapeutic agent comprises a C₂₀-C₂₂ polyunsaturated fatty acid.

In some embodiments, the therapeutic agent comprises a methylene-interrupted polyene.

In some embodiments, the methylene-interrupted polyene comprises a polyunsaturated Omega-3 fatty acid, a polyunsaturated Omega-6 fatty acid, or a polyunsaturated Omega-9 fatty acid. In some embodiments, the polyunsaturated Omega-3 fatty acid comprises alpha-linolenic acid (ALA)(all-cis-9,12,15-octadecatrienoic acid), stearidonic acid (SDA)(all-cis-6,9,12,15,-octadecatetraenoic acid), eicosatrienoic acid (ETE)(all-cis-11,14,17-eicosatrienoic acid), eicosatetraenoic acid (ETA)(all-cis-8,11,14,17-eicosatetraenoic acid), eicosapentaenoic acid (EPA, Timnodonic acid)(all-cis-5,8,11,14,17-eicosapentaenoic acid), heneicosapentaenoic acid (HPA)(all-cis-6,9,12,15,18-heneicosapentaenoic acid), docosapentaenoic acid (DPA, Clupanodonic acid)(all-cis-7,10,13,16,19-docosapentaenoic acid), docosahexaenoic acid (DHA, Cervonic acid)(all-cis-4,7,10,13,16,19-docosahexaenoic acid), tetracosapentaenoic acid (all-cis-9,12,15,18,21-tetracosapentaenoic acid), or tetracosahexaenoic acid (Nisinic acid)(all-cis-6,9,12,15,18,21-tetracosahexaenoic acid). In some embodiments, the polyunsaturated Omega-6 fatty acid comprises linoleic acid (all-cis-9,12-octadecadienoic acid), gamma-linolenic acid (GLA)(all-cis-6,9,12-octadecatrienoic acid), eicosadienoic acid (all-cis-11,14-eicosadienoic acid), dihomo-gamma-linolenic acid (DGLA)(all-cis-8,11,14-eicosatrienoic acid), arachidonic acid (AA)(all-cis-5,8,11,14-eicosatetraenoic acid), docosadienoic acid (all-cis-13,16-docosadienoic acid), adrenic acid (all-cis-7,10,13,16-docosatetraenoic acid), docosapentaenoic acid (Osbond acid)(all-cis-4,7,10,13,16-docosapentaenoic acid), tetracosatetraenoic acid (all-cis-9,12,15,18-tetracosatetraenoic acid), or tetracosapentaenoic acid (all-cis-6,9,12,15,18-tetracosapentaenoic acid). In some embodiments, the polyunsaturated Omega-9 fatty acid comprises mead acid (all-cis-5,8,11-eicosatrienoic acid).

In some embodiments, the therapeutic agent comprises a conjugated fatty acid. In some embodiments, the conjugated fatty acid comprises rumenic acid (9Z,11E-octadeca-9,11-dienoic acid or 10E,12Z-octadeca-10,12-dienoic acid), α-calendic acid (8E,10E,12Z-octadecatrienoic acid), β-calendic acid (8E,10E,12E-octadecatrienoic acid), jacaric acid (8Z,10E,12Z-octadecatrienoic acid), α-eleostearic acid (9Z,11E,13E-octadeca-9,11,13-trienoic acid), 0-eleostearic acid (9E,11E,13E-octadeca-9,11,13-trienoic acid), catalpic acid (9Z,11Z,13E-octadeca-9,11,13-trienoic acid), punicic acid (9Z,11E,13Z-octadeca-9,11,13-trienoic acid), rumelenic acid (9E,11Z,15E-octadeca-9,11,15-trienoic acid), α-parinaric acid (9E,11Z,13Z,15E-octadeca-9,11,13,15-tetraenoic acid), β-parinaric acid (all trans-octadeca-9,11,13,15-tetraenoic acid), or bosseopentaenoic acid (5Z,8Z,10E,12E,14Z-eicosanoic acid).

In some embodiments, the therapeutic agent comprises pinolenic acid ((5Z,9Z,12Z)-octadeca-5,9,12-trienoic acid) or podocarpic acid ((5Z,11Z,14Z)-eicosa-5,11,14-trienoic acid.

In some embodiments, the therapeutic agent comprises nicotinamide, curcumin, or a combination thereof.

In some embodiments, the therapeutic agent comprises a vector comprising a polynucleotide encoding ELOVL2 or a functionally-active fragment thereof. In some embodiments, the polynucleotide encodes a polypeptide comprising at least 80%, 85%, 90%, 95%, or 99% sequence identity to SEQ ID NO: 1. In some embodiments, the vector comprises a viral vector. In some embodiments, the viral vector comprises an adeno-associated virus (AAV)-based vector. In some embodiments, the viral vector comprises an adenovirus-based vector, an alphavirus-based vector, a herpesvirus-based vector, a retrovirus-based vector, a lentivirus-based vector, or a vaccinia virus-based vector.

In some embodiments, the vector comprises a promoter, an enhancer, an inverted terminal repeats (ITR), a capsid, polyadenylation signal, a signal sequence, or a combination thereof.

In some embodiments, the therapeutic agent comprises a vector comprising a polynucleotide encoding KLF14 or a functionally-active fragment thereof.

In some embodiments, the polynucleotide encodes a polypeptide comprising at least 80%, 85%, 90%, 95%, or 99% sequence identity to SEQ ID NO: 2.

In some embodiments, the composition further comprises a pharmaceutically acceptable carrier.

In some embodiments, the composition is formulated for oral administration.

In some embodiments, the aging phenotype comprises hair loss, a decrease in bone density, a decrease in endurance, a decrease in muscle strength, or a neurodegeneration.

In some embodiments, the subject is human.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1I illustrate Elovl2 as a metabolic gene that serves as an aging marker. FIG. 1A shows a correlation between DNA methylation of genes and aging. The sites of interest are marked. FIG. 1B shows MeDip-qPCR and qPCR of Elovl2 in human fibroblasts. Error bars, standard error of the mean (SEM). FIG. 1C shows DNA methylation level on the CpG island in intron 1 (CGI-I1) of Elovl2 in the brain and liver of 129/sv mice. Error bars, SEM. Different letters (a, b), p<0.05. FIG. 1D shows Beta-galactosidase (β-GAL) staining on young (p5) human fibroblasts with or without hydrogen peroxide (H₂O₂) treatment. Scale bar, 100 μm. FIG. 1E shows qPCR showing the transcriptional changes of cellular senescence markers in normal and H₂O₂ treated human fibroblasts. Error bars, SEM. *, p<0.05. FIG. 1F shows MeDip-qPCR of Elovl2 in H₂O₂ treated human fibroblasts. Error bars, SEM. FIG. 1G shows co-immunoprecipitation on human fibroblasts with or without H₂O₂ treatment. FIG. 1H shows Western blotting showing that the H₂O₂-induced accumulation of DNMTs to the chromatins was CHD4-dependent. FIG. 1I shows the recruitment of CHD4 and 5mC on DNA damage sites of cells irradiated with a 450 nm laser. Scale bar, 5 μm.

FIG. 2A-FIG. 2H illustrate deletion of Elovl2 causes severely accelerated aging phenotype and metabolic dysfunctions in mice. FIG. 2A shows hair loss in young (8 month) Elovl2 knockout (−/− Y) but not wild type (WT-Y) 129/sv mice. FIG. 2B shows micro-computed tomography (micro-CT) showing bone volume/total volume (BV/TV) and trabecular thickness (Tb. Th.) in the femur of mice. Error bars, SEM. Different letters (a, b), p<0.05. FIG. 2C shows Open-field test results in different groups of 129/sv mice. Error bars, SEM. Different letters (a, b, c), p<0.05. FIG. 2D shows hematoxylin and eosin staining and pathological section analysis of liver tissue. Green cycles show the abnormal structures. Different letters (a, b), p<0.05. Scale bar, 100 μm. FIG. 2E shows heat-map of fatty acid species in the liver, brain and plasma of 129/sv mice. FIG. 2F shows Oil Red O (ORO) staining of liver. Error bars, SEM. Different letters (a, b, c), p<0.05. Scale bar, 100 μm. FIG. 2G shows ultrasound test results. Error bars, SEM. Different letters (a, b, c, d), p<0.05. FIG. 2H shows glucose tolerance test (GTT) and insulin tolerance test (ITT) results. Error bars, SEM. *, p<0.05.

FIG. 3A-FIG. 3G illustrate depletion of Elovl2 leading to chronic inflammation, cellular senescence and adult stem cell exhaustion. FIG. 3A shows inflammatory factors levels in blood. Error bars, SEM. Different letters (a, b, c), p<0.05. FIG. 3B shows Western blotting and qPCR of TNF-α and MCP-1. Error bars, SEM and *, p<0.05. FIG. 3C shows Masson's trichrome staining on liver. Error bars, SEM. Different letters (a, b, c), p<0.05. Scale bar, 100 μm. FIG. 3D shows hair follicles and intestines stained with the epithelial progenitor cell markers. Scale bar, 100 μm. Error bars, SEM. Different letters (a, b, c), p<0.05. FIG. 3E shows auto-fluorescence images of the fundus drusen; propidium staining (red) showing different layers of retina, including ganglion cell layer (GCL), inner plexiform layer (OPL), outer nuclear layer (ONL), layers of rods and cones (RCL) and retina pigment epithelium (RPE); TUNEL staining (green) showed apoptosis. Error bars, SEM. Different letters (a, b, c), p<0.05. Scale bar, 800 μm for Auto-fluorescence images; 100 μm for slices. FIG. 3F shows the thickness of the NRL layer. Error bars, SEM. Different letters (a, b, c), p<0.05. FIG. 3G shows visual function analysis. Error bars, SEM. *, p<0.05.

FIG. 4A-FIG. 4I illustrate Elovl2 deficiency led to ER stress and mitochondrial dysfunction. FIG. 4A shows enriched gene sets of differentially expressed genes in −/− Y samples. The horizontal axis represents the differentially expressed genes in −/− Y compared to WT-O samples which were ranked as either up- or down-regulated in −/− Y and marked in red and blue, respectively. The normalized enrichment score (NES) and “““false””” discovery rate (FDR) are marked. FIG. 4B shows the Gene Ontology terms enriched in up- or down-regulated genes in −/− Y. FIG. 4C shows expression pattern of genes in aging-related pathways. FIG. 4D shows HSPA5 staining in liver. Error bars, SEM. Different letters (a, b), p<0.05. FIG. 4E shows Western blotting and qPCR for the markers of ER stress. Error bars, SEM. *, p<0.05. FIG. 4F shows Mitochondrial function analysis. Error bars, SEM. *, p<0.05. FIG. 4G shows the Seahorse XF mitochondrion stress test. Error bars, SEM. *, p<0.05. FIG. 4H shows Western blotting and RT-qPCR of HIF1α, ANT2, UCP2 and COX5b. Error bars, SEM. Different letters (a, b), p<0.05. FIG. 4I shows the Seahorse XF glycolysis test. Error bars, SEM. *, p<0.05.

FIG. 5A FIG. 5J illustrate AMD phenotype induced by depletion of Elovl2 in human RPE cells. FIG. 5A shows β-galactosidase staining on human RPE cells with no treatment (blank, blue) or Elovl2 knockdown (KE, purple). FIG. 5B shows cell doubling time analysis. Error bars, SEM. *, p<0.05. FIG. 5C and FIG. 5D show Western blotting (FIG. 5C) and qPCR (FIG. 5D) of senescence and AMD marker in blank and KE cells. *, p<0.05. FIG. 5E shows the expression pattern of genes in ER stress- and cellular senescence-related pathways. FIG. 5F shows the Gene Ontology terms enriched in up-regulated genes in KE RPE cells. FIG. 5G shows mitochondrial function analysis. Error bars, SEM. *, p<0.05. FIG. 5H shows mitoSOX staining results. Scale bar, 100 μm. FIG. 5I shows Immunofluorescence of blank and KE RPE cells treated with nicotinamide riboside (Ni) with VEGF and Aj3 antibodies. Scale bar, 100 μm. FIG. 5J shows qPCR analysis of cells undergoing various treatments for cellular senescence, AMD and mitochondrial function associated genes. Error bars, SEM. Different letters (a, b, c), p<0.05.

FIG. 6A FIG. 6H illustrate Elovl2 as a metabolic gene that serves as an aging marker. FIG. 6A shows the schematic of the structure of Elovl2 gene. FIG. 6B shows the DNA methylation levels measured by bisulfite-sequencing on the CpG islands of intron 1 (I1) and exon 3, 4, and 8 (E3, 4, 8) of Elovl2 in brain and liver of 129/sv and ICR mice. The error bars represent standard error of the mean (SEM). Different letters (a, b) indicate p<0.05. FIG. 6C shows the DNA methylation level on CGi-I1 in brain and liver of mice at the age of 1 week or 18 months. Methylation level was significantly higher in brain and liver of the 18 M mice. FIG. 6D shows Elovl2 expression level in brain, liver and testis of mice at the age of 1 week or 18 months. It showed that Elovl2 expression level decrease along with age. FIG. 6E shows Beta-galactosidase (β-GAL) staining on young (p5) and old (p38) human fibroblast cells. Scale bar=100 μm. FIG. 6F shows representative statistical chart of cell proliferation (cell number doubling time) of human fibroblast cells with or without hydrogen peroxide (H₂O₂) treatment. FIG. 6G shows co-immunoprecipitation results of human fibroblast cells with or without H₂O₂ treatment. Antibody of CHD4, G9a and Ezh2 were used. It showed CDH4 can recruit G9a and Ezh2. FIG. 6H shows Elovl2 expression level significantly decreased after H₂O₂ treatment in groups where DNMT1, DNMT3A or DNMT3B but not CHD4 were knockdown.

FIG. 7A FIG. 7H illustrates deletion of Elovl2 causes dramatic acceleration of aging in mice. FIG. 7A shows the design of sgRNA and genotype of mice in different groups. Figure discloses SEQ ID NOS 4-6, respectively, in order of appearance. FIG. 7B shows Western blotting showing the expression of ELOVL2 in different organs of WT or —/mice. FIG. 7C shows representative images showing the hair loss of WT-O or −/− mice. The bone volume/total volume (BV/TV), trabecular thickness (Tb. Th.), trabecular number (Tb. Nu.) and trabecular spacing (Tb. Sp.) of WT-Y, WT-O, and −/−Y was measured by Micro-CT. The error bars represent SEM. Different letters (a, b, c) indicate p<0.05. (D & E)−/− Y mice displayed reductions in endurance (FIG. 7D) and muscle strength (FIG. 7E). The error bars represent SEM. Different letters (a, b, c) indicate p<0.05. FIG. 7F shows the Morris Water Maze test with platforms at different spots of the pool has shown spatial learning and memory of mice in WT-Y, WT-O, and −/−Y (n=20). Despite of the platform location, it showed spatial learning and memory of WT-O and −/− mice were significantly decreased. The error bars represent SEM. Different letters (a, b, c) indicate p<0.05. FIG. 7G and FIG. 7H show representative images of hematoxylin and eosin staining and pathological section analysis of kidney and sciatic nerve; green cycles show the abnormal structures. Scale bar=100 μm. Different letters (a, b) indicate p<0.05.

FIG. 8A-FIG. 8E illustrates multiple metabolic disturbances that were found in Elovl2 KO mice. FIG. 8A shows the schematics showing the roles of ELOVL families in lipid metabolism. FIG. 8B shows the heat map of fatty acid species, saturated fatty acids (SFAs), mono-unsaturated fatty acids (MUFAs) and poly-unsaturated fatty acids (PUFAs) in liver, brain and plasma of ICR mice. FIG. 8C shows an exemplary glucose tolerance test (GTT)(left) and an exemplary insulin tolerance test (ITT)(right) for mice on regular diet (Corn oil) and PUFAs adding diet (Fish oil). For GTT, ICR Mice were injected with 1 g/kg glucose at time zero after 16 hr overnight fasting and serum glucose was measured for up to 2 h (n=12 per group). For ITT, ICR mice were injected with 1.6 U/kg insulin at time zero after 5 hr fasting from the onset of the light cycle (n=12 per group). The error bars represent SEM. *, p<0.05. (D). Distance, velocity and turn count of different groups of 129/sv mice and ICR mice fed with either corn oil or fish oil in the open-field test. FIG. 8E shows representative images of hematoxylin and eosin staining and pathological section analysis of liver and kidney from WT-Y, −/− Y fed with corn oil supplemented diet and −/− Y fed with fish oil supplemented diet.

FIG. 9A-FIG. 9F illustrates depletion of Elovl2 in mice leading to chronic inflammation and a decline in the function of eye and brain. FIG. 9A shows an ELISA test on inflammatory factors in blood samples from different groups of ICR mice. The error bars represent SEM. Different letters (a, b, c) indicate p<0.05. FIG. 9B shows ERG and VEP recording methodology analysis showed a decline of eye function. The error bars represent SEM. Different letters (a, b, c) indicate p<0.05. FIG. 9C shows Fundus Autofluorescence Imaging showed the appearance of drusen in −/− Y mice. FIG. 9D shows magnetic resonance imaging (MRI) analysis on the cerebral cortex and hippocampus. It revealed a dramatic abnormity in −/− Y and WT-O mice. The error bars represent SEM. Different letters (a, b, c) indicate p<0.05. FIG. 9E and FIG. 9F show gene ontology analysis of the RNA-Seq data revealed disfunction in the brain of −/−Y mice.

FIG. 10A-FIG. 10F illustrates Elovl2 ablation leading to severe oxidative damage at the cellular level. FIG. 10A shows enriched gene sets of differentially expressed genes in −/− Y samples. The horizontal axis represents the differentially expressed genes in −/− Y compared to high fat diet (HFD) mouse samples which were ranked as either up- or down-regulated in −/− Y. The normalized enrichment score (NES) and false discovery rate (FDR) are marked. FIG. 10B shows expression pattern of genes in ER stress pathways in WT-Y and −/− Y liver samples. FIG. 10C shows qPCR results verified the RNA-Seq data. The error bars represent SEM. * indicates p<0.05. FIG. 10D shows MitoSOX staining and γH2A.X staining showed sever oxidative damage in mitochondria and nuclei respectively in −/− Y mice. The error bars represent SEM. Different letters (a, b) indicate p<0.05. FIG. 10E shows ELISA test on oxidative damage factors in liver samples from different groups. It showed that the oxidative damage affecting proteins (AOPP), lipids (MDA), and RNA (8-OHG). The error bars represent SEM. Different letters (a, b) indicate p<0.05. FIG. 10F shows higher cellular senescent markers were detected by western blotting and qPCR in −/− Y and WT-O mice. The error bars represent SEM. Different letters (a, b) indicate p<0.05.

FIG. 11A-FIG. 11D illustrates AMD phenotype as induced by Elovl2 deficiency. FIG. 11A and FIG. 11B show qPCR (FIG. 11A) and Western blotting (FIG. 11B) results showing ELOVL2 expressions in blank and KE human RPE cells. FIG. 11C shows a heatmap showing the cluster of cellular senescence-associated genes with variation between blank and KE human RPE cells. FIG. 11D shows RNA-seq data which revealed that the upregulated genes and downregulated genes in KE human RPE cells.

FIG. 12 illustrates a schematic of a model for age-related DNA methylation mediated accelerated aging process.

FIG. 13 illustrates a correlation and function of genes with aging marker CpG sites. The P-value of each gene was ranked from the largest to the smallest, and the function of the top genes is showed on the right.

FIG. 14 illustrates an exemplary AAV-based ELOVL2 construct described herein.

### DETAILED DESCRIPTION OF THE DISCLOSURE

ELOVL fatty acid elongase 2 (ELOVL2) encodes a transmembrane protein involved in catalyzing the rate-limiting step of the long-chain fatty acids elongation cycle. In some instances, the methylation level or methylation status of ELOVL2 correlates to a decrease in ELOVL2 protein expression. For example, the methylation state or level of ELOVL2 increases as a subject ages and conversely, the protein expression also decreases.

In some instances, an increase in ELOVL2 methylation status and a decrease in ELOVL2 expression have been correlated to an increase in aging process. For example, a depletion of ELOVL2 aborts polyunsaturated fatty acids (PUFAs) synthesis and accelerates aging in a mouse model through switch cellular energetic metabolism to a glycolysis state, increase of endoplasmic reticulum (ER) stress, and mitochondrial dysfunction.

In some embodiments, disclosed herein is a method of retarding and/or reversing the aging phenotype by increasing the expression of ELOVL2 and/or by modulating the PUFA synthesis pathway.

### Methods of Use

In some embodiments, disclosed herein is a method of reducing or slowing-down an aging phenotype in a subject in need thereof, comprising administering to the subject a composition comprising a therapeutic agent that reduces or slows-down the aging phenotype. In some instances, the therapeutic agent comprises a C₁₈-C₂₈ polyunsaturated fatty acid. In some cases, the therapeutic agent comprises a C₂₀-C₂₈ polyunsaturated fatty acid, a C₂₀-C₂₂ polyunsaturated fatty acid, a C₂₂-C₂₈ polyunsaturated fatty acid, or a C₂₂-C₂₄ polyunsaturated fatty acid. In some cases, the therapeutic agent comprises a C₁₈ polyunsaturated fatty acid. In some cases, the therapeutic agent comprises a C20 polyunsaturated fatty acid. In some cases, the therapeutic agent comprises a C₂₁ polyunsaturated fatty acid. In some cases, the therapeutic agent comprises a C₂₂ polyunsaturated fatty acid. In some cases, the therapeutic agent comprises a C₂₄ polyunsaturated fatty acid. In some cases, the therapeutic agent comprises a C₂₆ polyunsaturated fatty acid. In some cases, the therapeutic agent comprises a C₂₈ polyunsaturated fatty acid.

In some instances, the therapeutic agent comprises a methylene-interrupted polyene. In some cases, the methylene-interrupted polyene comprises a polyunsaturated Omega-3 fatty acid, a polyunsaturated Omega-6 fatty acid, or a polyunsaturated Omega-9 fatty acid. Exemplary polyunsaturated Omega-3 fatty acids include, but are not limited to, alpha-linolenic acid (ALA) (all-cis-9,12,15-octadecatrienoic acid), stearidonic acid (SDA)(all-cis-6,9,12,15,-octadecatetraenoic acid), eicosatrienoic acid (ETE)(all-cis-11,14,17-eicosatrienoic acid), eicosatetraenoic acid (ETA)(all-cis-8,11,14,17-eicosatetraenoic acid), eicosapentaenoic acid (EPA, Timnodonic acid)(all-cis-5,8,11,14,17-eicosapentaenoic acid), heneicosapentaenoic acid (HPA)(all-cis-6,9,12,15,18-heneicosapentaenoic acid), docosapentaenoic acid (DPA, Clupanodonic acid)(all-cis-7,10,13,16,19-docosapentaenoic acid), docosahexaenoic acid (DHA, Cervonic acid)(all-cis-4,7,10,13,16,19-docosahexaenoic acid), tetracosapentaenoic acid (all-cis-9,12,15,18,21-tetracosapentaenoic acid), or tetracosahexaenoic acid (Nisinic acid)(all-cis-6,9,12,15,18,21-tetracosahexaenoic acid).

Exemplary polyunsaturated Omega-6 fatty acids include, but are not limited to, linoleic acid (all-cis-9,12-octadecadienoic acid), gamma-linolenic acid (GLA)(all-cis-6,9,12-octadecatrienoic acid), eicosadienoic acid (all-cis-11,14-eicosadienoic acid), dihomo-gamma-linolenic acid (DGLA)(all-cis-8,11,14-eicosatrienoic acid), arachidonic acid (AA)(all-cis-5,8,11,14-eicosatetraenoic acid), docosadienoic acid (all-cis-13,16-docosadienoic acid), adrenic acid (all-cis-7,10,13,16-docosatetraenoic acid), docosapentaenoic acid (Osbond acid)(all-cis-4,7,10,13,16-docosapentaenoic acid), tetracosatetraenoic acid (all-cis-9,12,15,18-tetracosatetraenoic acid), or tetracosapentaenoic acid (all-cis-6,9,12,15,18-tetracosapentaenoic acid).

Exemplary polyunsaturated Omega-9 fatty acids include, but are not limited to, mead acid (all-cis-5,8,11-eicosatrienoic acid).

In some instances, the therapeutic agent comprises a polyunsaturated Omega-3 fatty acid. In some cases, the therapeutic agent comprises alpha-linolenic acid (ALA)(all-cis-9,12,15-octadecatrienoic acid), stearidonic acid (SDA)(all-cis-6,9,12,15,-octadecatetraenoic acid), eicosatrienoic acid (ETE)(all-cis-11,14,17-eicosatrienoic acid), eicosatetraenoic acid (ETA)(all-cis-8,11,14,17-eicosatetraenoic acid), eicosapentaenoic acid (EPA, Timnodonic acid)(all-cis-5,8,11,14,17-eicosapentaenoic acid), heneicosapentaenoic acid (HPA)(all-cis-6,9,12,15,18-heneicosapentaenoic acid), docosapentaenoic acid (DPA, Clupanodonic acid)(all-cis-7,10,13,16,19-docosapentaenoic acid), docosahexaenoic acid (DHA, Cervonic acid)(all-cis-4,7,10,13,16,19-docosahexaenoic acid), tetracosapentaenoic acid (all-cis-9,12,15,18,21-tetracosapentaenoic acid), or tetracosahexaenoic acid (Nisinic acid)(all-cis-6,9,12,15,18,21-tetracosahexaenoic acid).

In some instances, the therapeutic agent comprises a polyunsaturated Omega-6 fatty acid. In some cases, the therapeutic agent comprises linoleic acid (all-cis-9,12-octadecadienoic acid), gamma-linolenic acid (GLA)(all-cis-6,9,12-octadecatrienoic acid), eicosadienoic acid (all-cis-11,14-eicosadienoic acid), dihomo-gamma-linolenic acid (DGLA)(all-cis-8,11,14-eicosatrienoic acid), arachidonic acid (AA)(all-cis-5,8,11,14-eicosatetraenoic acid), docosadienoic acid (all-cis-13,16-docosadienoic acid), adrenic acid (all-cis-7,10,13,16-docosatetraenoic acid), docosapentaenoic acid (Osbond acid)(all-cis-4,7,10,13,16-docosapentaenoic acid), tetracosatetraenoic acid (all-cis-9,12,15,18-tetracosatetraenoic acid), or tetracosapentaenoic acid (all-cis-6,9,12,15,18-tetracosapentaenoic acid).

In some instances, the therapeutic agent comprises a polyunsaturated Omega-9 fatty acid. In some cases, the therapeutic agent comprises mead acid (all-cis-5,8,11-eicosatrienoic acid).

In some instances, the therapeutic agent comprises a conjugated fatty acid. Exemplary conjugated fatty acids include, but are not limited to, rumenic acid (9Z,11E-octadeca-9,11-dienoic acid or 10E,12Z-octadeca-10,12-dienoic acid), α-calendic acid (8E,10E,12Z-octadecatrienoic acid), β-calendic acid (8E,10E,12E-octadecatrienoic acid), jacaric acid (8Z,10E,12Z-octadecatrienoic acid), α-eleostearic acid (9Z,11E,13E-octadeca-9,11,13-trienoic acid), β-eleostearic acid (9E,11E,13E-octadeca-9,11,13-trienoic acid), catalpic acid (9Z, 11Z,13E-octadeca-9,11,13-trienoic acid), punicic acid (9Z,11E,13Z-octadeca-9,11,13-trienoic acid), rumelenic acid (9E,11Z,15E-octadeca-9,11,15-trienoic acid), α-parinaric acid (9E,11Z,13Z,15E-octadeca-9,11,13,15-tetraenoic acid), β-parinaric acid (all trans-octadeca-9,11,13,15-tetraenoic acid), or bosseopentaenoic acid (5Z,8Z,10E,12E,14Z-eicosanoic acid). In some cases, the therapeutic agent comprises rumenic acid (9Z,11E-octadeca-9,11-dienoic acid or 10E,12Z-octadeca-10,12-dienoic acid), α-calendic acid (8E,10E,12Z-octadecatrienoic acid), 3-calendic acid (8E,10E,12E-octadecatrienoic acid), jacaric acid (8Z,10E,12Z-octadecatrienoic acid), α-eleostearic acid (9Z,11E,13E-octadeca-9,11,13-trienoic acid), β-eleostearic acid (9E,11E,13E-octadeca-9,11,13-trienoic acid), catalpic acid (9Z,11Z,13E-octadeca-9,11,13-trienoic acid), punicic acid (9Z,11E,13Z-octadeca-9,11,13-trienoic acid), rumelenic acid (9E,11Z,15E-octadeca-9,11,15-trienoic acid), α-parinaric acid (9E,11Z,13Z,15E-octadeca-9,11,13,15-tetraenoic acid), β-parinaric acid (all trans-octadeca-9,11,13,15-tetraenoic acid), or bosseopentaenoic acid (5Z,8Z,10E,12E,14Z-eicosanoic acid).

In some instances, the therapeutic agent comprises pinolenic acid ((5Z,9Z,12Z)-octadeca-5,9,12-trienoic acid) or podocarpic acid ((5Z,11Z,14Z)-eicosa-5,11,14-trienoic acid.

In some instances, the therapeutic agent comprises nicotinamide, curcumin, or a combination thereof.

In some embodiments, the therapeutic agent comprises a vector comprising a polynucleotide encoding ELOVL2 or a functionally-active fragment thereof. In some instance, the therapeutic agent is further formulated as a composition for upregulating ELOVL2 expression. In such instances, the method comprises treating a subject in need thereof, which comprises administering to the subject a composition comprising the vector comprising a polynucleotide encoding ELOVL2 or a functionally-active fragment thereof.

In some instance, the polynucleotide encodes a polypeptide comprising at least 80%, 85%, 90%, 91%, 92%, 93%, 94%, 95%, 96%, 97%, 98%, or 99% sequence identity to SEQ ID NO: 1. In some cases, the polynucleotide encodes a polypeptide comprising at least 80%, 85%, 90%, 95%, or 99% sequence identity to SEQ ID NO: 1. In some cases, the polynucleotide encodes a polypeptide comprising at least 80% sequence identity to SEQ ID NO: 1. In some cases, the polynucleotide encodes a polypeptide comprising at least 85% sequence identity to SEQ ID NO: 1. In some cases, the polynucleotide encodes a polypeptide comprising at least 90% sequence identity to SEQ ID NO: 1. In some cases, the polynucleotide encodes a polypeptide comprising at least 91% sequence identity to SEQ ID NO: 1. In some cases, the polynucleotide encodes a polypeptide comprising at least 92% sequence identity to SEQ ID NO: 1. In some cases, the polynucleotide encodes a polypeptide comprising at least 93% sequence identity to SEQ ID NO: 1. In some cases, the polynucleotide encodes a polypeptide comprising at least 94% sequence identity to SEQ ID NO: 1. In some cases, the polynucleotide encodes a polypeptide comprising at least 95% sequence identity to SEQ ID NO: 1. In some cases, the polynucleotide encodes a polypeptide comprising at least 96% sequence identity to SEQ ID NO: 1. In some cases, the polynucleotide encodes a polypeptide comprising at least 97% sequence identity to SEQ ID NO: 1. In some cases, the polynucleotide encodes a polypeptide comprising at least 98% sequence identity to SEQ ID NO: 1. In some cases, the polynucleotide encodes a polypeptide comprising at least 99% sequence identity to SEQ ID NO: 1. In some cases, the polynucleotide encodes a polypeptide comprising 100% sequence identity to SEQ ID NO: 1. In some cases, the polynucleotide encodes a polypeptide consists of the sequence set forth in SEQ ID NO: 1.

In some instances, the vector comprises a viral vector. In some cases, the viral vector comprises an adeno-associated virus (AAV)-based vector. Exemplary AAV-based vectors include, but are not limited, to AAV-based vector of serotype 1 (AAV1), AAV-based vector of serotype 2 (AAV2), AAV-based vector of serotype 3 (AAV3), AAV-based vector of serotype 4 (AAV4), AAV-based vector of serotype 5 (AAV5), AAV-based vector of serotype 6 (AAV6), AAV-based vector of serotype 7 (AAV7), AAV-based vector of serotype 8 (AAV8), AAV-based vector of serotype 9 (AAV9), or a humanized AAV-based vector.

In some cases, the vector comprises AAV-based vector of serotype 1 (AAV1), AAV-based vector of serotype 2 (AAV2), AAV-based vector of serotype 3 (AAV3), AAV-based vector of serotype 4 (AAV4), AAV-based vector of serotype 5 (AAV5), AAV-based vector of serotype 6 (AAV6), AAV-based vector of serotype 7 (AAV7), AAV-based vector of serotype 8 (AAV8), AAV-based vector of serotype 9 (AAV9), or a humanized AAV-based vector.

In some cases, the viral vector comprises an adenovirus-based vector, an alphavirus-based vector, a herpesvirus-based vector, a retrovirus-based vector, a lentivirus-based vector, or a vaccinia virus-based vector.

In some instances, the vector comprises a cell or tissue-specific promoter operatively linked to the polynucleotide described above. In some cases, the cell or tissue-specific promoter is an endogenous promotor specific to the cell type of interest. In other cases, the cell or tissue-specific promoter is an exogenous promotor specific to the cell type of interest.

In some instances, the vector comprises a microbial promoter. In some cases, the microbial promoter comprises SV40. In other cases, the microbial promoter comprises cytomegalovirus (CMV) immediate-early promoter.

In some instances, the vector comprises an enhancer, an inverted terminal repeats (ITR), a capsid, polyadenylation signal, a signal sequence, or a combination thereof.

In some embodiments, the vector comprises a selectable marker. In some instances, the selectable marker comprises a polynucleotide encoding a fluorescent protein. In some cases, the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyan 1, Midori-Ishi Cyan, TagCFP, mTFPl, enhanced yellow fluorescent protein (EYFP), Topaz, MCitrine, YPet, TagYFP, PhiYFP, ZsYellow 1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRedl, mRaspberry, dKeima-Tandem, mPlum, or AQ143.

In some embodiments, the vector comprises a polynucleotide encoding an elongation factor 1-alpha (EF1α).

In some embodiments, the vector comprises a polynucleotide encoding a Klarsicht, ANC-1, Syne Homology (KASH) domain.

In some embodiments, the therapeutic agent comprises a vector comprising a polynucleotide encoding KLF14 or a functionally-active fragment thereof. Kruppel-like factor 14 (KLF14), also known as basic transcription element-binding protein 5 (BTEB5), encodes a member of the Kruppel-like family of transcription factors. In some instances, KLF14 protein regulates the transcription of TGFβR11 and is a master regulator of gene expression in adipose tissue. In some instances, the methylation level or methylation status of KLF14 correlates to a decrease in KLF14 protein expression.

In some instance, the therapeutic agent is formulated as a composition for upregulating KLF14 expression. In such instances, the method comprises treating a subject in need thereof, which comprises administering to the subject a composition comprising the vector comprising a polynucleotide encoding KLF14 or a functionally-active fragment thereof. In some cases, the polynucleotide encodes a polypeptide comprising at least 80%, 85%, 90%, 95%, or 99% sequence identity to SEQ ID NO: 2.

In some embodiments, the composition comprises an active agent that inhibits activation of chromodomain-helicase-DNA-binding protein 4 (CHD4).

In some embodiments, a reduced ELOVL2 expression level correlates with an increase in accumulation of a plurality of fatty acids with less than 22 carbon chains. In some instances, the plurality of fatty acids comprises saturated fatty acids, monounsaturated fatty acids, or a combination thereof.

In some embodiments, an elevated expression of ELOVL2 reduces or slows-down an aging phenotype. In some cases, the aging phenotype comprises hair loss, a decrease in bone density, a decrease in endurance, a decrease in muscle strength, or neurodegeneration. In some cases, an elevated expression of ELOVL2 reduces or slows-down hair loss, a decrease in bone density, a decrease in endurance, a decrease in muscle strength, neurodegeneration, or a combination thereof.

In some embodiments, an elevated expression of ELOVL2 treats an aging-related disease or condition. In some instances, the aging-related disease or condition is age-related macular degeneration (AMD). Age-related macular degeneration (also known as macular degeneration, AMD, or ARMD) is a worsening of vision that results in either a blurred vision or no vision at the center of the visual field. In some instances, oxidative stress, lipid molecule accumulation, and inflammation contribute to the development of AMD. In some cases, a composition comprising a vector described above treat AMD. In other cases, a composition comprising a vector described above reduces and/or slows-downs the progression of AMD.

In some instances, the aging-related disease or indication is a metabolic disease or condition. In such instances, the metabolic disease or condition is diabetes (diabetes mellitus, DM). In some cases, diabetes is type 1 diabetes, type 2 diabetes, type 3 diabetes, type 4 diabetes, double diabetes, latent autoimmune diabetes (LAD), gestational diabetes, neonatal diabetes mellitus (NDM), maturity onset diabetes of the young (MODY), Wolfram syndrome, Alstrom syndrome, prediabetes, or diabetes insipidus. Type 2 diabetes, also called non-insulin dependent diabetes, is the most common type of diabetes accounting for 95% of all diabetes cases. In some cases, type 2 diabetes is caused by a combination of factors, including insulin resistance due to pancreatic beta cell dysfunction, which in turn leads to high blood glucose levels. In some cases, increased glucagon levels stimulate the liver to produce an abnormal amount of unneeded glucose, which contributes to high blood glucose levels.

Type 1 diabetes, also called insulin-dependent diabetes, comprises about 5% to 10% of all diabetes cases. Type 1 diabetes is an autoimmune disease where T cells attack and destroy insulin-producing beta cells in the pancreas. In some embodiments, Type 1 diabetes is caused by genetic and environmental factors.

In some embodiments, the term double diabetes is used to describe patients diagnosed with both type 1 and 2 diabetes.

Type 4 diabetes is a recently discovered type of diabetes affecting about 20% of diabetic patients age 65 and over. In some embodiments, type 4 diabetes is characterized by age-associated insulin resistance.

In some embodiments, type 3 diabetes is used as a term for Alzheimer's disease resulting in insulin resistance in the brain.

LAD, also known as slow onset type 1 diabetes, is a slow developing form of type 1 diabetes where diagnosis frequently occurs after age 30. In some embodiments, LAD is further classified into latent autoimmune diabetes in adults (LADA) or latent autoimmune diabetes in the young (LADY) or latent autoimmune diabetes in children (LADC).

Prediabetes, also known as borderline diabetes, is a precursor stage to diabetes mellitus. In some cases, prediabetes is characterized by abnormal OGTT, fasting plasma glucose test, and hemoglobin AlC test results. In some embodiments, prediabetes is further classified into impaired fasting glycaemia or impaired fasting glucose (IFG) and impaired glucose tolerance (IGT). IFG is a condition in which blood glucose levels are higher than normal levels, but not elevated enough to be diagnosed as diabetes mellitus. IGT is a pre-diabetic state of abnormal blood glucose levels associated with insulin resistance and increased risk of cardiovascular pathology.

In some cases, a composition comprising a vector described above treat a metabolic disease or condition. In some cases, a composition comprising a vector described above treat diabetes.

In some instance, the therapeutic agent is formulated as a composition for upregulating the expression of one or more additional genes. In such instances, the method comprises treating a subject in need thereof, which comprises administering to the subject a composition comprising the vector comprising a polynucleotide encoding the one or more additional genes or a functionally-active fragment thereof. In some cases, the one or more additional genes are selected from Slc6a4, Sst, Hdac4, Nefm, Calbl, Il4i1, Grin2c, Chga, Grm2, Neurod1, Ardb1, Dio3, Ghsr, Avpr1α, Cadps2, Gria2, Irs2, Smad2, Htr7, Sypl2, Mad1l1, or Vgf. In some instance, the therapeutic agent is formulated as a composition for upregulating the expression of Slc6a4, Sst, Hdac4, Nefm, Calbl, Il4i1, Grin2c, Chga, Grm2, Neurod1, Ardb1, Dio3, Ghsr, Avpr1α, Cadps2, Gria2, Irs2, Smad2, Htr7, Sypl2, Mad1l1, Vgf or a combination thereof. In some instance, the therapeutic agent is formulated as a composition for upregulating the expression of Slc6a4. In some instance, the therapeutic agent is formulated as a composition for upregulating the expression of Sst. In some instance, the therapeutic agent is formulated as a composition for upregulating the expression of Hdac4. In some instance, the therapeutic agent is formulated as a composition for upregulating the expression of Nefm. In some instance, the therapeutic agent is formulated as a composition for upregulating the expression of Calbl. In some instance, the therapeutic agent is formulated as a composition for upregulating the expression of Il4il. In some instance, the therapeutic agent is formulated as a composition for upregulating the expression of Grin2c. In some instance, the therapeutic agent is formulated as a composition for upregulating the expression of Chga. In some instance, the therapeutic agent is formulated as a composition for upregulating the expression of Grm2. In some instance, the therapeutic agent is formulated as a composition for upregulating the expression of Neurod1. In some instance, the therapeutic agent is formulated as a composition for upregulating the expression of Ardb1. In some instance, the therapeutic agent is formulated as a composition for upregulating the expression of Dio3. In some instance, the therapeutic agent is formulated as a composition for upregulating the expression of Ghsr. In some instance, the therapeutic agent is formulated as a composition for upregulating the expression of Avprla. In some instance, the therapeutic agent is formulated as a composition for upregulating the expression of Cadps2. In some instance, the therapeutic agent is formulated as a composition for upregulating the expression of Gria2. In some instance, the therapeutic agent is formulated as a composition for upregulating the expression of Irs2. In some instance, the therapeutic agent is formulated as a composition for upregulating the expression of Smad2. In some instance, the therapeutic agent is formulated as a composition for upregulating the expression of Htr7. In some instance, the therapeutic agent is formulated as a composition for upregulating the expression of Sypl2. In some instance, the therapeutic agent is formulated as a composition for upregulating the expression of Mad1l1. In some instance, the therapeutic agent is formulated as a composition for upregulating the expression of Vgf.

In some instance, the therapeutic agent is formulated as a composition for upregulating the expression of Elovl2 in combination with one or more of Slc6a4, Sst, Hdac4, Nefm, Calbl, Il4i1, Grin2c, Chga, Grm2, Neurod1, Ardb1, Dio3, Ghsr, Avprla, Cadps2, Gria2, Irs2, Smad2, Htr7, Sypl2, Mad1l1, or Vgf.

In some instance, the therapeutic agent is formulated as a composition for upregulating the expression of Elovl2 and Klfl4 in combination with one or more of Slc6a4, Sst, Hdac4, Nefm, Calbl, Il4i1, Grin2c, Chga, Grm2, Neurod1, Ardb1, Dio3, Ghsr, Avprla, Cadps2, Gria2, Irs2, Smad2, Htr7, Sypl2, Mad1l1, or Vgf.

In some instance, the therapeutic agent is formulated as a composition for upregulating the expression of Klfl4 in combination with one or more of Slc6a4, Sst, Hdac4, Nefm, Calbl, Il4il, Grin2c, Chga, Grm2, Neurod1, Ardb1, Dio3, Ghsr, Avprla, Cadps2, Gria2, Irs2, Smad2, Htr7, Sypl2, Mad1l1, or Vgf.

### Pharmaceutical Compositions and Formulations

In some embodiments, a composition comprising a therapeutic agent or an active agent (e.g., a C₁₈-C₂₈ polyunsaturated fatty acid or a vector comprising a polynucleotide encoding ELOVL2 or a functionally-active fragment thereof) are administered to a subject by multiple administration routes, including but not limited to, parenteral (e.g., intravenous, subcutaneous, intramuscular), oral, intranasal, buccal, rectal, or transdermal administration routes. In some instances, the composition (e.g., a pharmaceutical composition) is formulated for oral administration. In some instances, the composition (e.g., a pharmaceutical composition) is formulated for parenteral administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab_O_Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, a therapeutic agent described herein is administered for one or more times a day. In some embodiments, a therapeutic agent described herein is administered once per day, twice per day, three times per day or more. In some cases, a therapeutic agent described herein is administered daily, every day, every alternate day, five days a week, once a week, every other week, two weeks per month, three weeks per month, once a month, twice a month, three times per month, or more. In some cases, a therapeutic agent described herein is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/of” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

A “site” corresponds to a single site, which in some cases is a single base position or a group of correlated base positions, e.g., a CpG site. A “locus” corresponds to a region that includes multiple sites. In some instances, a locus includes one site.

The term “vector” is used herein to refer to a nucleic acid molecule having nucleotide sequences that enable its replication in a host cell. In some instances, vectors comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Selection of these and other common vector elements are conventional and many such sequences are derived from commercially available vectors. See e.g., Sambrook et al. (1989) Molecular Cloning: A Laboratory Manual. Cold Spring Harbor Laboratory Press, Cold Spring Harbor, and references cited therein.

The term “operatively linked”, as used herein, refers to a functional combination between, e.g., a promoter region and a polynucleotide (e.g., encoding ELOVL2 protein) such that the transcription of the polynucleotide is controlled and regulated by the promoter region.

As used herein, a “functional” protein is one that retains at least one biological activity normally associated with that protein. Preferably, a “functional” protein retains all of the activities possessed by the unmodified protein. A “non-functional” protein is one that exhibits essentially no detectable biological activity normally associated with the protein (e.g., at most, only an insignificant amount).

### EXAMPLES

These examples are provided for illustrative purposes only and not to limit the scope of the claims provided herein.

### Example 1

Aging is characterized by gradual increase of vulnerability to pathologies loss of molecular fidelity and progressively decline in tissue and organ function. Several studies have shown that aging is further correlated to epigenetic alterations. Epigenetic alteration encompasses post-translational modification on genome and further integrates environmental signals to regulate gene expression and downstream cellular processes in aging. Indeed, methylation level of CpG sites that correlate with biological age have been mapped (Broer, L., and van Duijn, C. M. (2015). GWAS and Meta-Analysis in Aging/Longevity. Adv Exp Med Biol 847, 107-125; Hannum, et al. (2013). Genome-wide methylation profiles reveal quantitative views of human aging rates. Mol Cell 49, 359-367; and Stubbs, et al. (2017). Multi-tissue DNA methylation age predictor in mouse. Genome Biol 18, 68). Several of the age-related methylation sites have also been correlated to metabolism associated genes (Hannum, et al. (2013). Genome-wide methylation profiles reveal quantitative views of human aging rates. Mol Cell 49, 359-367; Zbiec-Piekarska, et al. (2015). Examination of DNA methylation status of the ELOVL2 marker may be useful for human age prediction in forensic science. Forensic Sci Int Genet 14, 161-167). ELOVL fatty acid elongase 2 (Elovl2) is involved in the synthesis of PUFAs such as DHA and EPA and a lack of DHA and EPA have been observed leading to age-related diseases in both animal models and human. See, e.g., Pauter, et al. (2017). Both maternal and offspring Elovl2 genotypes determine systemic DHA levels in perinatal mice. J Lipid Res 58, 111-123; and Zadravec, et al. (2011). ELOVL2 controls the level of n−6 28:5 and 30:5 fatty acids in testis, a prerequisite for male fertility and sperm maturation in mice. J Lipid Res 52, 245-255) In some cases, PUFAs are involved in anti-inflammation, energy generation, and homeostasis of lipid metabolism (Buckley, et al. (2014). Proresolving lipid mediators and mechanisms in the resolution of acute inflammation. Immunity 40, 315-327; Hennebelle, et al. (2014). Ageing and apoE change DHA homeostasis: relevance to age-related cognitive decline. Proc Nutr Soc 73, 80-86; and Serhan, C. N. (2014). Pro-resolving lipid mediators are leads for resolution physiology. Nature 510, 92-101). The following example examines epigenetic alteration with respect to PUFA synthesis and contribution of aging in mammals.

### Elovl2+/−& −I− Mouse

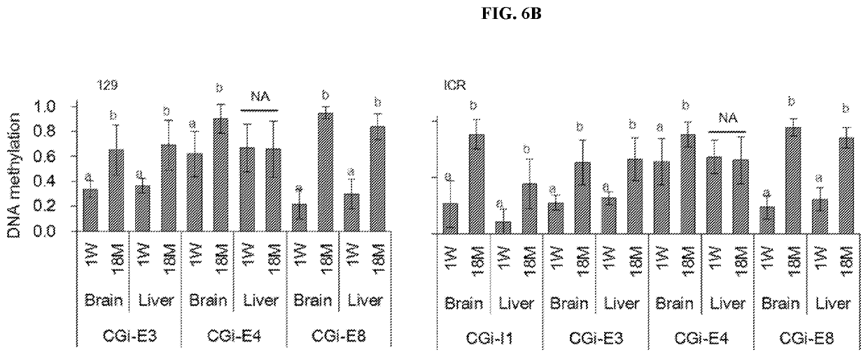

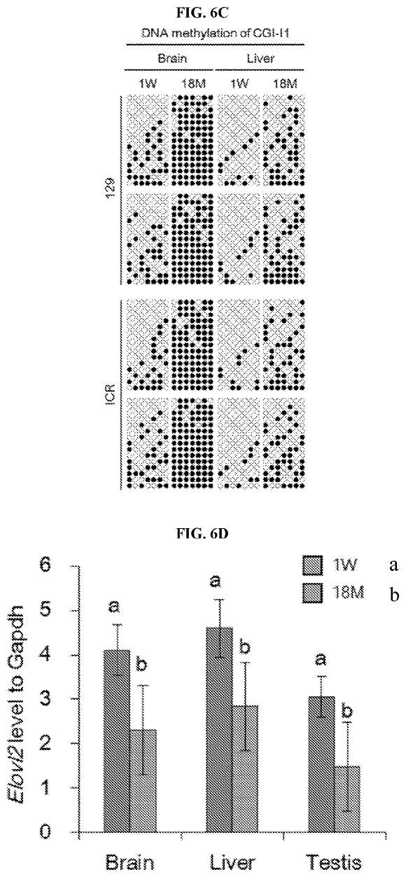

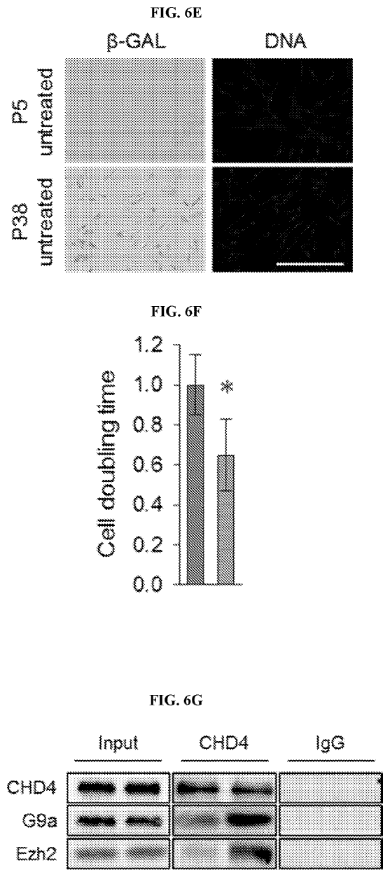

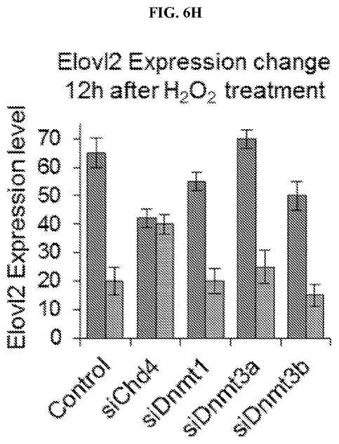

All mouse experiments were performed in accordance with ARRIVE guidelines and regulations. Zygotes were collected from 6-week-old ICR superovulated female mice crossed with ICR males, at post human chorionic gonadotropin (phCG) injection 21 hours. Cas9 mRNA and sgRNAs targeting Elovl2 exon 3 (FIG. 6, 20 ng/μL for each) were injected at phCG 25 hours. The Cas9 mRNA were in vitro transcribed with mMESSAGEmMACHINE® T7 ULTRA Kit (Ambion, AMB1345-5), and sgRNAs were synthesized by in vitro transcription using MEGAshortscript™ Kit (Ambion, AM1354).

### Cell Culture and Drug Treatment

The human RPE cells were primarily derived from healthy donors. The human fibroblast cells are purchased from ATCC (WI-38, ATCC® CCL-75) human fibroblasts were maintained in Dulbecco's Modified Eagle Medium (DMEM) supplemented with 10% fetal bovine serum and 50 U/ml penicillin-streptomycin. Human RPE cells were cultured with DMEM/F-12 (1:1) supplemented with 10% fetal bovine serum and 50 U/ml penicillin-streptomycin.

For Elovl2 knockdown, lentivector (pLL3.7-shElovl2) exogenously expressing shRNA (5′-TGGTGGTACTATTTCTCCAAA-3′)(SEQ ID NO:3) was made and transfected into 293T cells (ATCC) using Lipofectamin 3000 reagent (Thermo Fisher) to obtain lentivirus. To build the Elovl2-knockdown cell line, human RPE cells (seeded a night ahead in a concentration of 5*10≡cells/well) in a 6-well plate were infected with the lentivirus containing Elovl2-shRNA for 24 h, followed by 24 h of equilibration. Drug treatments were performed at the time of 48 h post-infection. To rescue the Elovl2 knockdown cells, 10 μg/ml curcumin (Sigma, C7727) or 5 mM nicotinamide (Sigma, N0636) or DMSO were added in the cell culture medium and the cells were treated for 48 h.

RNA Extraction, Reverse Transcription, and qPCR

RNA was extracted by RNeasy Mini Kit (QIAGEN, 74104) and the RNase-Free DNase Set (QIAGEN, 79254) was used to ensure no DNA contamination. Reverse transcription was performed by High Capacity cDNA Reverse Transcription Kit (ABI, 4368814). SYBR-qPCR was performed by Power SYBR® Green PCR Master Mix (ABI, 4367659). All primers were designed using PrimerPremier5 and synthesized from Integrated DNA Technologies.

### Western Blot

Fresh tissue suspension was obtained by mechanical trituration. Human fibroblast cells and human RPE cells were collected and lysed in Pierce IP Lysis Buffer (Pierce, 87787), supplied with Protease Inhibitor Cocktail (Pierce, 78441) and sodium orthovanadate (Sigma, S6508) on ice for 30 min. After 13,000 g centrifugation for 10 min at 4° C., supernatants were collected and mixed with 30 μL sample buffer (10 mL; 1.25 mL 0.5 M-pH 6.8-Tris-HCl, 2.5 mL glycerin, 2 mL 10% SDS, 200 μL 0.5% bromophenol blue, 3.55 mL H2O, and 0.5 mL 0-mercaptoethanol) and incubated for 5 minutes in boiling water. The samples were separated on SDS-PAGE with a 5% stacking gel (10 mL; 5.7 mL ddH₂O, 2.5 mL 1.5 M pH 6.8 Tris-HCl, 1.7 mL 30% acrylamide (acryl:bis amyl=29:1), 100 μI, 10% SDS, 50 μL 10% ammonium persulfate, and 10 μL TEMED) and a 10% separating gel (10 mL; 4.1 mL ddH2O, 2.5 mL 1.5 M pH 8.8 Tris-HCl, 3.3 mL 30% acrylamide (acryl:bis acryl=29:1), 100 μL 10% SDS, 50 μL 10% ammonium persulfate, and 5 μL TEMED) at 100 V for 1 hour, and then electrophoretically transferred onto a nitrocellulose membrane at 200 mA for 1 hour at 4° C. Membranes were blocked in TBST buffer (10 mM Tris, 150 mM NaCl, 0.1% Tween 20, pH 7.4) containing 3% BSA (Sigma, B2064), for 1 hour at RT and then incubated with primary antibody, diluted in TBST containing 1% BSA, overnight at 4° C. After three washes for 10 minutes each in TBST, the membrane was incubated for 1 hour at RT with the secondary antibody diluted in TBST. After three washes for 10 minutes each, the signals were detected using ECL and films.

### Co-Immunoprecipitation (Co-IP)

Co-immunoprecipitation of chromatin bound proteins was performed on human fibroblast cells and human RPE cells with the Nuclear Complex Co-IP kit (Active Motif, 54001) following instructions of manufacturer. Briefly, cells were lysed in hypotonic buffer and the nuclear extracts (tight chromatin) were gained via centrifugation. Extracts were digested with enzymatic shearing cocktails and incubate overnight with anti-CHD4 antibody (Sigma, SAB4200107) at 4° C. following by the pull-down with protein A/G magnetic beads (Thermo, 88802). The pull-down products were washed, denatured and eluted according to the manufacturer's instruction.

### Micro-Computed Tomography Bone Density Analysis

Micro-CT was performed using the Inveon MM system (Siemens, Munich, Germany) Images with 8.82 μm pixel size were acquired under 60 kV of voltage, 300 μA of current and 1,500 ms of exposure time during the 3600 rotational step. 2000 slices of images with voxel size of 8.82 μm×8.82 μm×8.82 μm were acquired. 3D reconstruction was performed using multimodal 3D visualization software (Inveon Research Workplace, SIEMENS, Munich, Germany).

### Open Field Test

The apparatus consisted of a square-shaped arena (600×600 mm2, length×width) constructed by blue plastic, and illuminated evenly at 15 1 ux50. Test mice were placed facing the center of one wall and allowed to explore the apparatus for 10 min. The open field was subdivided into two virtual concentric squares (center region and all region). The distance and the velocity spent in all regions were calculated.

### Morris Water Maze

The water maze was built in a black tank filled with water at room temperature. During the training period, mice were trained to find a fixed platform submerged at a constant position below the water surface in one of the quadrants. The mice were placed at four settled spots in the tank and allowed to find a foothold. If a mouse failed to reach the hidden platform in 90 s, it was led to it manually and stayed for 15 s. The mice were trained for 5 days with 5 consecutive trials per day. The natant trajectory was recorded and analyzed using image analysis to calculate the path length, swim velocity and number of turns that mice made. For the spatial learning evaluation, the difference between the path length of day 1 and day 5 was compared. For the spatial memory ability test, the platform was removed from the tank and the mice were released and freely swim in the maze for 90 s. The path length, swim velocity and turn numbers were measured.

### Accelerating Rotarod Test

The test was performed using a rotating cylinder system, following instruction of the manufacturer. Briefly, mice were placed on a rotating rod that rotated from 4 to 40 r.p.m. for 5 min. The time until falling off or losing balance was recorded. For three consecutive days, each mouse was tested for three trials per day with 30 min interval between trials.

### Grip Strength Test

The grip strength test was performed on mice using a grip strength measuring system. The mice were allowed to grasp a sensing bar attached to a force strength meter. After reaching the bar with both paws symmetrically, the mice were gently pulled away until the grasp broke. The mean value in five consecutive trails was taken as the score. Results were normalized with body weight (g).

### Histopathological Analysis and Oil Red O Staining

Upon being harvested from animals, murine tissues were fixed in 4% paraformaldehyde (PFA), embedded in paraffin and sectioned. HE staining was performed following standard procedures. Pathological parameters, including necrosis, infiltration of lymphocytes and monocytes, vascular formulation and fibrosis were evaluated.

For the oil red O (ORO) staining, liver tissues were fixed in 4% PFA and equilibrated with 30% sucrose, following with optimal cutting temperature (OCT) compound embedding, snap-freezing and sectioning. ORO staining was performed on cryosections using Lipid (Oil Red O) Staining Kit (Sigma, MAK194). The adipocyte size and numbers were quantified.

### Lipidomic Analysis

For the purpose of lipidomic analysis, liver and brain of mice were harvested and immediately stored in liquid nitrogen until extraction. Plasma were acquired from freshly collected blood by centrifugation and immediately frozen. Lipid extraction was performed following the standard procedure of chloroform-methanol method. The gas chromatography-mass spectrum analysis was performed on the fatty acid extracts according to instructions of the manufacturer. Briefly, a standard curve was built with SPLASH® Lipidomix® Mass Spec Standard (SPLASH, 330707). Levels of saturated fatty acids, monounsaturated fatty acids and poly-unsaturated fatty acids were evaluated.

### Glucose Tolerance Test and Insulin Tolerance Test

During GTT, an intraperitoneal injection of glucose (Sigma, G7528) with a single dose of 2 g/kg body weight was performed in 6 h-fasted mice. Blood samples were collected from the tail vein before glucose injection (0 min) and at 15, 30, 60 and 120 min afterward. Blood glucose concentration was immediately measured by a glucose meter (ONETOUCH Ultra, Lifescan).

ITT was performed by intraperitoneal injection of insulin (0.75 IU/Kg, Aladdin, 12584-58-6). Blood glucose concentrations were measured before insulin injection (0 min) and 30, 60, 90 and 120 min after insulin injection. Blood samples were collected from mice tail vain and blood glucose concentration were immediately measured by a glucose meter (ONETOUCH Ultra, Lifescan).

### RNA-Seq Library Preparation and Data Processing

Total RNA was extracted from tissues or cultured cells by TRIzol reagent. For RNA-sequence library construction, the PolyA+tailed RNA purification was performed for each sample using mRNA purification kit. The cDNA library was generated with a Stranded mRNA-Seq Kit.

Sequencing was performed on an Illumina HiSeq 4000 platform with 150 bp paired-end-sequencing reactions. The RNA-sequence reads of each sample were mapped to the mouse mm9 or human hg19 genome assembly independently by the HISAT2 software using the annotated gene structures as templates. Default parameters of HISAT2 were used except with the options “—dta-cufflinks” and “—rna-strandness RF” opening (HISAT: a fast spliced aligner with low memory requirements. Nature Methods 12(4): 357-360; 2015). Reads with unique genome location were retained for gene expression calculation using Cufflinks (version 2.0.2) with the option “—GTF” (Transcript assembly and quantification by RNA-Seq reveals unannotated transcripts and isoform switching during cell differentiation, Nature Biotechnology, 28(5): 511-515; 2010).

The heatmap was produced by the heatmap.2 function of R. Gene ontology analysis of differentially expressed genes was analyzed by DAVID (Huang D W, Sherman B T, Lempicki R A. Systematic and integrative analysis of large gene lists using DAVID Bioinformatics Resources. Nature Protoc. 2009; 4(1):44-57, Huang D W, Sherman B T, Lempicki R A. Bioinformatics enrichment tools: paths toward the comprehensive functional analysis of large gene lists. Nucleic Acids Res. 2009; 37(1):1-13) and biological processes were selected based on P-values smaller than 0.05 and the FIGURES were produced by ggplot2. The gene set enrichment analysis was performed by GSEA (Gene set enrichment analysis: A knowledge-based approach for interpreting genome-wide expression profiles, PNAS, 102(43): 15545-15550; 2005) and the gene sets were constructed from the published data sets (Comprehensive transcriptional landscape of aging mouse liver, BMC Genomics. 16: 899; 2015; The metabolic response to a high-fat diet reveals obesity-prone and -resistant phenotypes in mice with distinct mRNA-seq transcriptome profiles. Int J Obes (Lond). 40:1452-1460; 2016).

### Immunofluorescence Staining and MitoSOX Staining

Murine tissue sample were freshly collected upon perfusion, followed by overnight PFA fixation and sucrose dehydration. Human fibroblast cells, human RPE cells and murine primary cells were fixed with 4% PFA for 1 h. To perform immunofluorescence, the slide was rinsed in PBS for 5 minutes, blocked in blocking buffer (PBS with 1% BSA, 0.1% Tween-20) for 20 minutes at room temperature (RT), and incubated with primary antibody in blocking buffer for 1 hour at RT. After 3 washes with 0.1% Tween-20 in PBS, the slide was incubated with secondary antibody in blocking buffer for 1 hour at RT. The slides were mounted with DAPI-Vectashield solution (Vector laboratories). Images were taken with a confocal microscope (LSM 780). To estimate the mitochondrial conditions, human RPE cells and murine primary cells were incubated with either MitoSOX (5 μM) according to manufacturer's instructions.

### Glycolysis Stress Test and Mito Stress Test

The glycolysis stress test and the mito stress test were performed on murine primary hepatocytes with Seahorse Bioscience XF Analyzer (Agilent Tech) following the instructions of manufacturer. Briefly, the murine hepatocytes were seed in the XF96 cell culture microplate (Seahorse Bioscience, 101085-004) with 100,000 cells per well. Ahead of the assays, the culture medium was replaced followed by 1 h incubation in 37° C. For the glycolysis stress test, cell culture medium was replaced by Seahorse XF Base medium (Seahorse Bioscience), supplemented with L-glutamine. During the assay, glucose, oligomycin and 2-deoxyglucose were added into each well sequentially, followed by mixing and measurements. For the mito stress test, culture medium was replaced by Seahorse XF Base medium supplemented with glucose, L-glutamine and pyruvate. During the assay, oligomycin, FCCP and rotenone were added into each well sequentially, followed by mixing and measurements. Mixture time, incubation time and the timeline of chemicals addition were determined based according to instructions of manufacturer.

### Quantification and Statistical Analysis

Statistical analyses were performed in R. Levels of significance were calculated with two tailed student's t-test. In all FIGURES: *, p-value <0.05; **, p-value <0.01.

Correlation Between Elovl2 Methylation and Expression Status with the Biological Aging Use

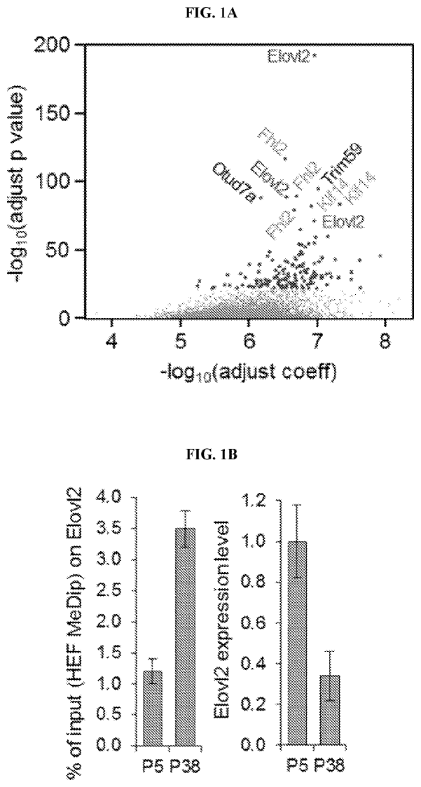

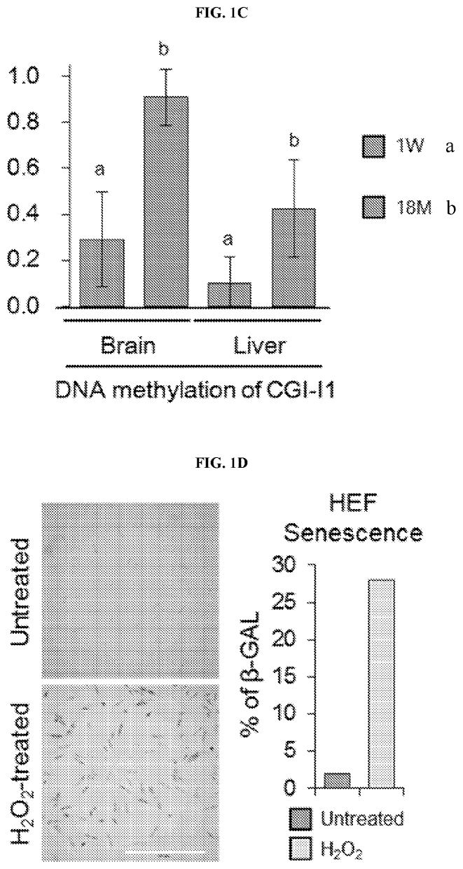

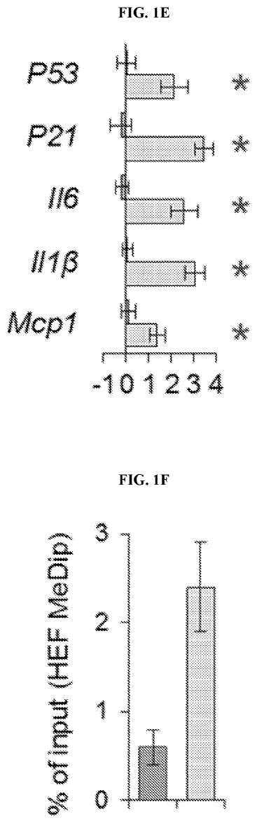

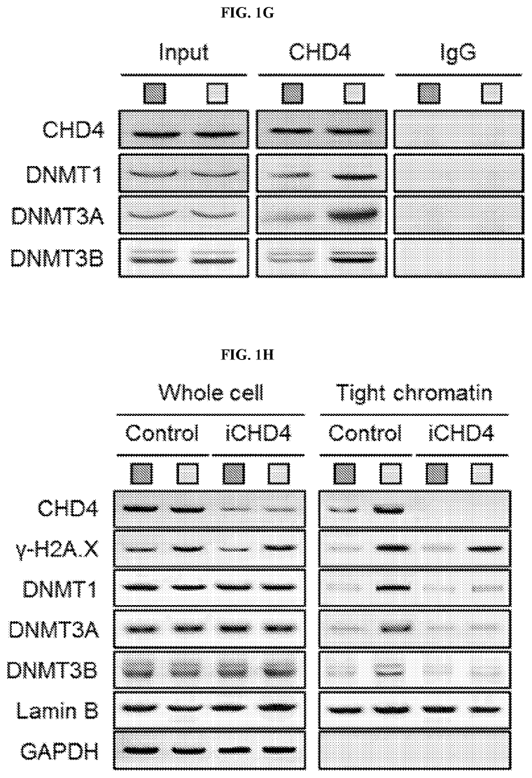

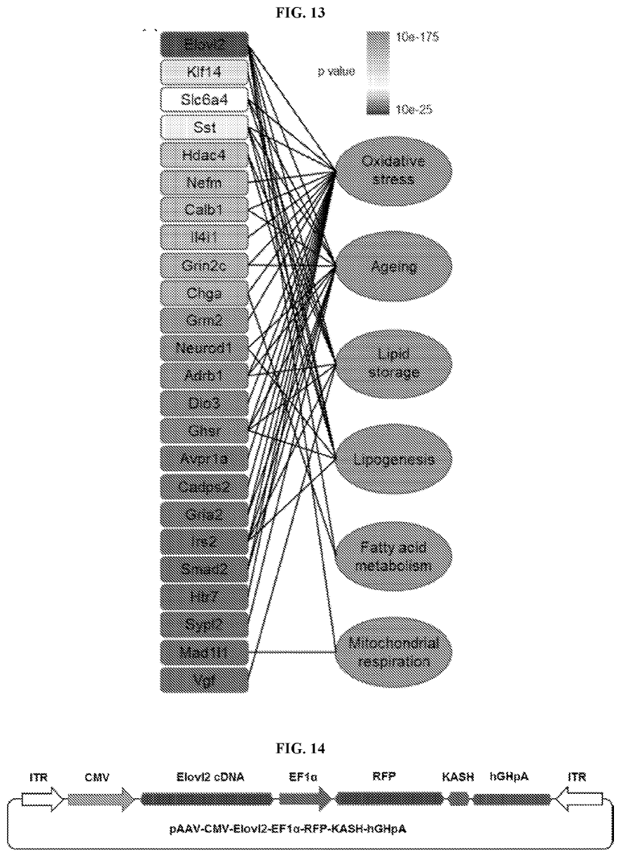

In some instances, methylation status increases with aging in human and mouse, and further serves as a biological age predictor. In some cases, the functions of these genes are further profiled and whether increased methylation status would affect gene transcriptional activities were explored. The dataset containing methylation profile of aging markers were analyzed. Functions of the top 20 genes are related to oxidative stress, aging, and lipid metabolism (FIG. 1A and FIG. 13). An increase in methylation on Elovl2 was observed in human fibroblasts accompanied by down-regulating Elovl2 expression level (FIG. 1).

In some cases, studies have shown that epigenetic clock also exists in mouse (Wang, et al., Epigenetic aging signatures in mice livers are slowed by dwarfism, calorie restriction and rapamycin treatment. Genome Biol 18, 57 (2017); Petkovich, et al., Using DNA Methylation Profiling to Evaluate Biological Age and Longevity Interventions. Cell Metab 25, 954-960 e956 (2017)). Methylation and expression of human age marker relating to aging in mouse was further examined.

Four CpG islands within the mouse Elovl2 gene were examined: CGI-I1 in the first intron, and CGI-E3, E4, and E8 in the 3^{rd}, 4^{th} and 8^{th} exons (FIG. 6A). It was found that the methylation of CGI-I1, E3, E4, and E8 in brain and liver increased with aging on 129/sv strains (FIG. 1C and FIGS. 6B-6C). qPCR analysis showed that the expression level of Elovl2 decreased with aging in 129 mice (FIG. 6D). The data was consistent on ICR strains (FIGS. 6B-6D). These results indicated that Elovl2 showed a conserved pattern of increased methylation and reduced expression in both human fibroblast cell model and mouse model.

### CHD4 Plays a Key Role in Mediation of DNA Methylation During Cellular Senescence and DNA Damage Repair Process.

CHD4, a NuRD component, recruits repressive chromatin proteins to sites of DNA damage repair, including DNA methyltransferases where it imposes de novo DNA methylation. In some instances, CHD4 comprises an oncogenic role of initiating and supporting tumor suppressor gene (TSG) silencing (Xia, et al. (2017). CHD4 Has Oncogenic Functions in Initiating and Maintaining Epigenetic Suppression of Multiple Tumor Suppressor Genes. Cancer Cell 31, 653-668 e657).

Next, how aging related methylation is mediated was examined. In some instances, accumulated DNA damage lead to cellular senescence in aging. As such, whether age-related DNA methylation was mediated by DNA damages and their repair process was examined. Induced cellular senescence with hydrogen peroxide (H₂O₂) has been used as an in vitro model for aging study. Here H₂O₂ treated human fibroblast cells were used as an aging model. H₂O₂ treated cells showed an increase of senescent markers consistent with the expression patterns in high-passage number (30-40 passages) cells (FIGS. 1D-1E and FIGS. 6E-6F). Increased methylation on biological aging markers (Elovl2) was observed in high-passage cells and H₂O₂ treated cells (FIG. 6F), which indicated that DNA methylation occurred in both H₂O₂-treated cells and high-passage number cells (FIG. 1i).

In some instances, studies have shown that CHD4 plays a role in DNA damage repair-mediated gene silencing in cancer cells (Xie, et al., CHD4 Has Oncogenic Functions in Initiating and Maintaining Epigenetic Suppression of Multiple Tumor Suppressor Genes. Cancer Cell 31, 653-668 e657 (2017)). To determine whether CHD4 plays a role in age-related DNA methylation, how CHD4 interacted with DNA methyltransferases and chromatin suppression modifiers was examined. Co-immunoprecipitation assays using human fibroblast cells showed CHD4 interacted with DNMT1, DNMT3A and DNMT3B after H₂O₂ treatment (FIG. 1H). Co-immunoprecipitation assays showed that CHD4 interacted significantly with DNMTs after H₂O₂ treatment (FIG. 1G). Western blotting showed that the binding of DNMTs to chromatins was promoted by H₂O₂ treatment (FIG. 1H, Tight chromatin, Control). Moreover, CHD4 knockdown diminished the binding of DNMT1, DNMT3A and DNMT3B to the chromatins (FIG. 1H, Tight chromatin, iCHD4), indicating that the H₂O₂-induced accumulation of DNMTs to the chromatins was CHD4-dependent. Besides DNMTs, other inhibitory histone modifiers such as EZH2 and G9a were also recruited by CHD4 (FIG. 6G). Additionally, by the time of 60 minutes after laser induced single strand breaks (SSBs) and double strand breaks (DSBs), endogenous endogenous CHD4, γH2a.X and increased 5mC signals were detected at the damage sites 60 minutes post laser-induced DNA damages, which were reduced upon CHD4 knock down (FIG. 1I).

Next, the expression of Elovl2 in human fibroblast cells before and after H₂O₂ treatment was examined. The expression of Elovl2 decreased after H₂O₂ treatment in groups with DNMT1, DNMT3A and DNMT3B knockdown but not in the CHD4 knockdown group (FIG. 6H), indicating that CHD4 mediated DNA methylation consequently downregulates the transcriptional activity of aging marker gene Elovl2. Overall, the results showed that after oxidative damage or DNA double/single strand break damage, CHD4 played a role in interacting and recruiting other epigenetic suppressors to add methylation to the damage sites in human cellular aging model.

### Deletion of Elovl2 Causes Severe Acceleration of Aging Phenotype in Mouse

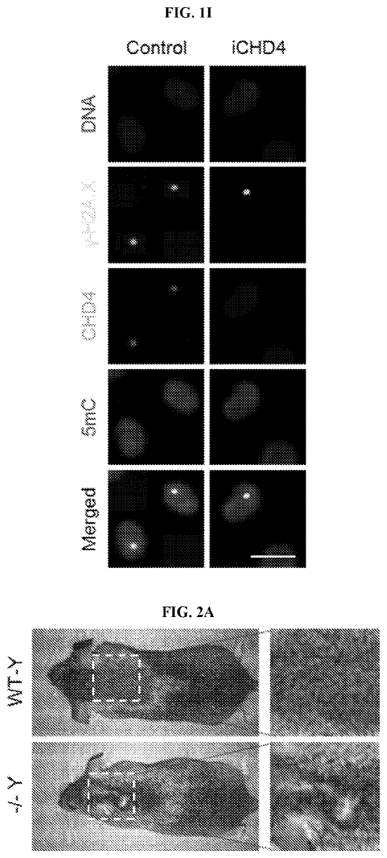

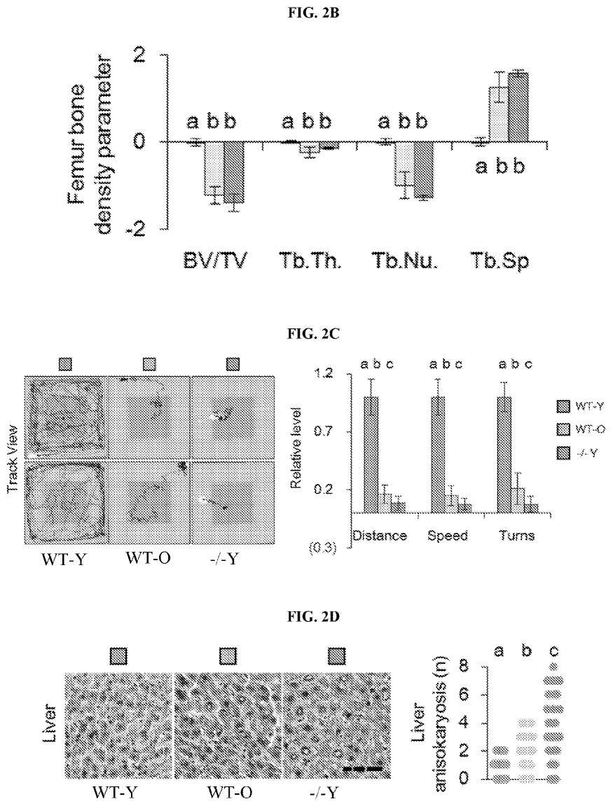

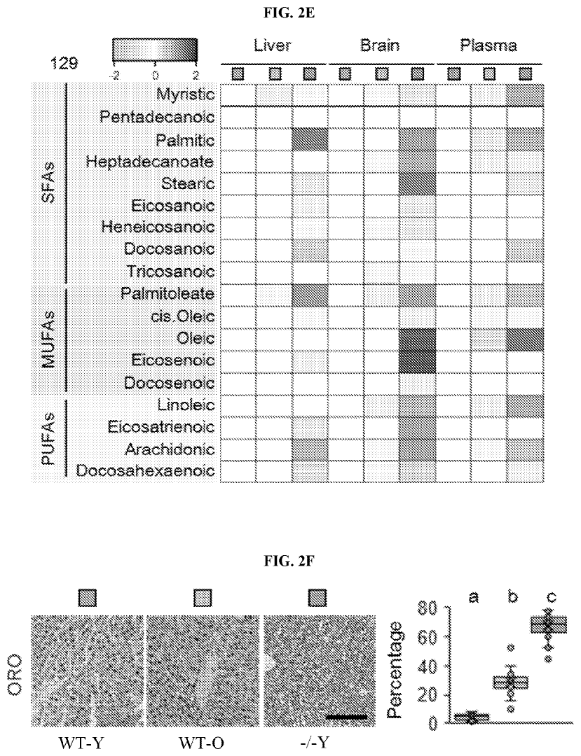

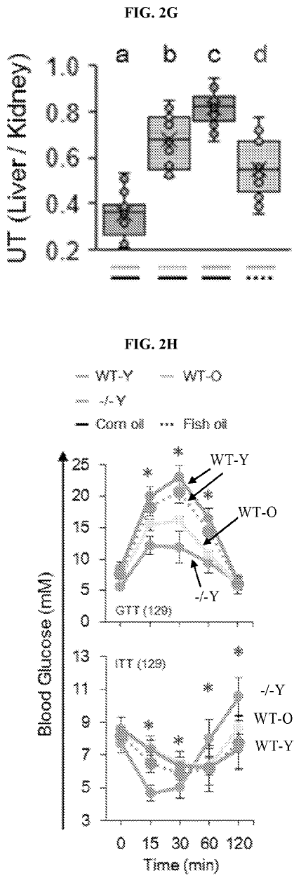

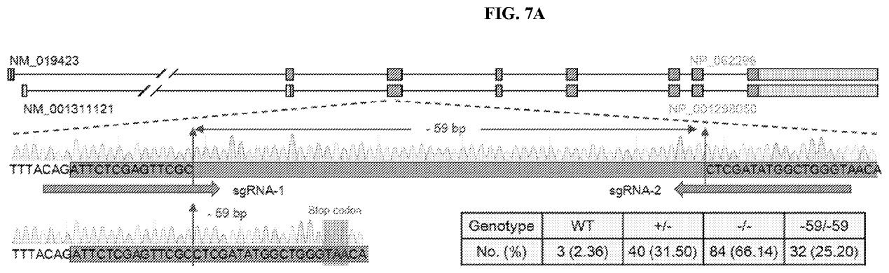

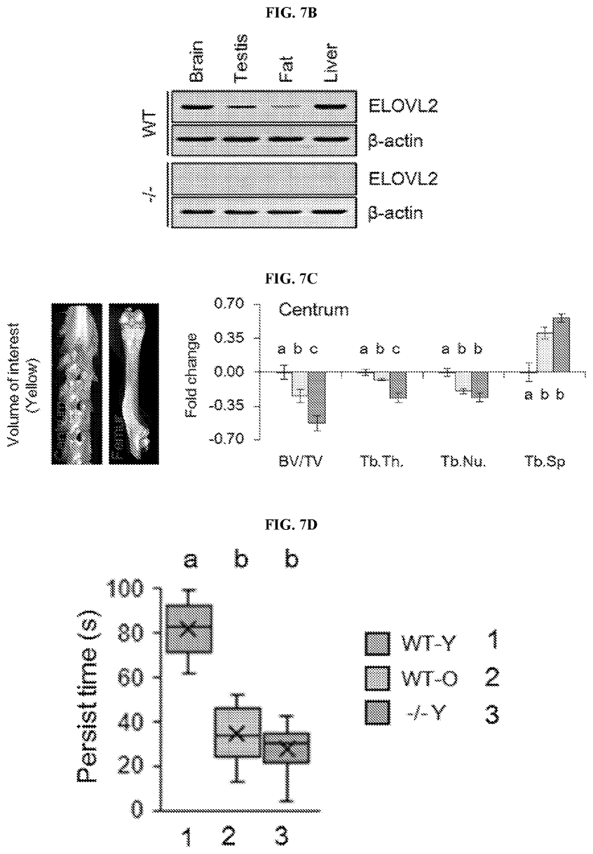

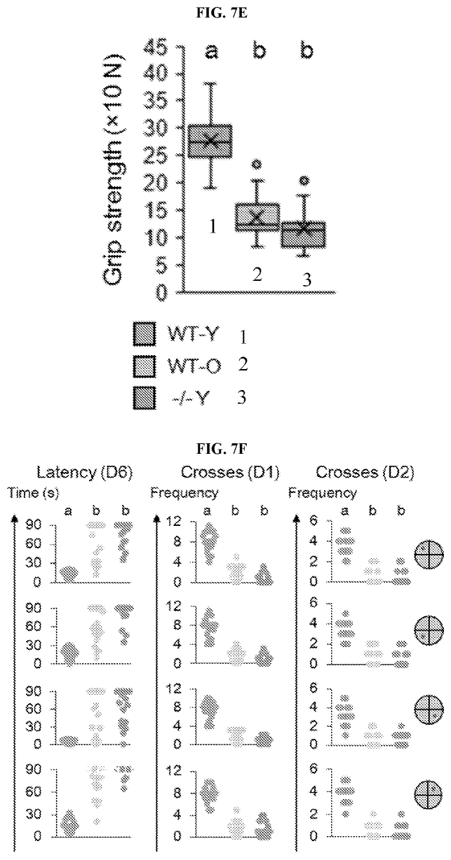

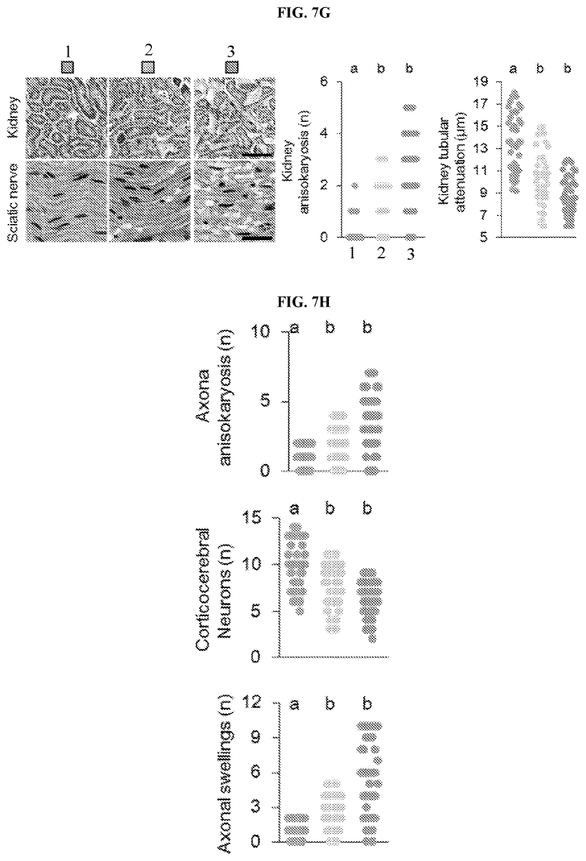

In order to examine the function of Elovl2 in aging, Elovl2 Knock out mice was generated with CRISPR-Cas9. SgRNAs were designed targeting the third exon (FIG. 7A). Knock out experiment were carried out on both 129/sv and ICR mice. In total 40 Elovl2+/− and 84 Elovl2+/− funder mouse were generated. In these Elovl2^{-/-} mice, 32 mice have a deletion of 59 bp in the third exon that led a stop codon. The experiments were carried on with these 59 bp deletion mouse because both Elovl2^{+/-} and Elovl2^{-/-} mice were infertile despite the gender or strain, which was not consistent with studies such as Zadravec, et al., ELOVL2 controls the level of n−6 28:5 and 30:5 fatty acids in testis, a prerequisite for male fertility and sperm maturation in mice. J Lipid Res 52, 245-255 (2011). Stringent Non-PUFA milk and ingredient-defined non-PUFA diet was used for the pups or adult mice to prevent the consumption of PUFAs from the food intake. Western blot showed that Elovl2 was completely depleted (FIG. 7B). Aging parameters were examined after 8 months (treated as young, Y) of growth with ingredient-defined non-PUFA diet. Elovl2^{-/-} young mice (−/− Y) showed series of aging-accelerated phenotypes, such as hair loss (FIG. 2A), decrease of bone density (FIG. 2B and FIG. 7C), endurance (FIG. 7D), and muscle strength (FIG. 7E). Since the progressive neurodegeneration often occurs with aging in human premature aging disease and animal models, a series of behavioral and motor functional test were performed. Open field behavior test showed −/− Y mice had decrease on movement distance, speed and the number of turns, which was similar to wild type old (WT-O, 20 months old) mice (FIG. 2C), revealing a decreased exploratory behavior and increased anxiety. Morris water maze test suggested decay in learning and memorizing ability in −/− Y mice (FIG. 7F). Moreover, aging-related histopathological phenotypes were detected in −/− Y and WT-O mice (FIG. 2D and FIGS. 7G-7H). Aging-related histopathological phenotypes were found in −/− Y and WT-O mice, such as liver anisokaryosis and intranuclear inclusions, kidney anisokaryosis, and severe axonal swellings in sciatic nerve (FIG. 2D and FIG. 7G).

### Elovl2 Deficiency Disturbs the Lipid and Energetic Metabolism

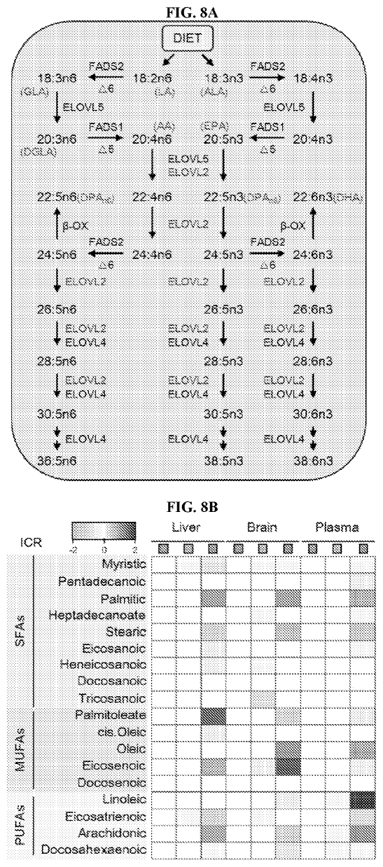

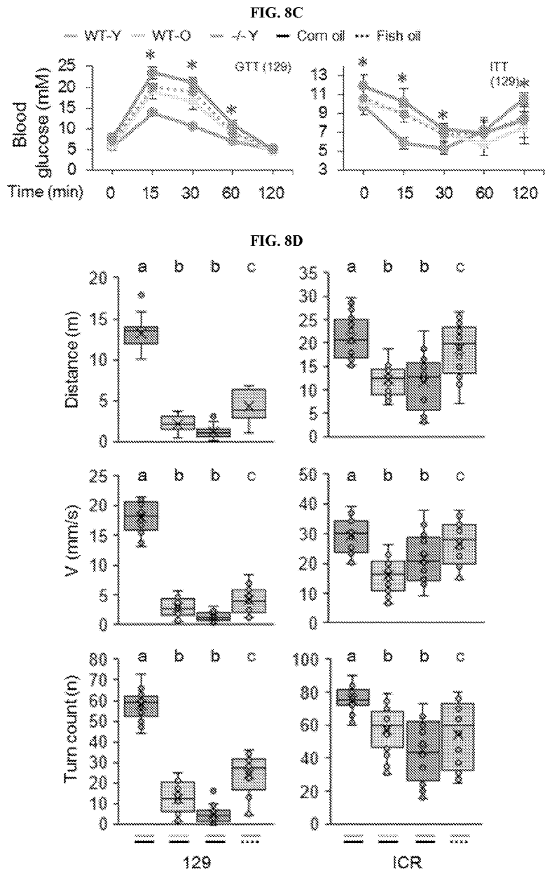

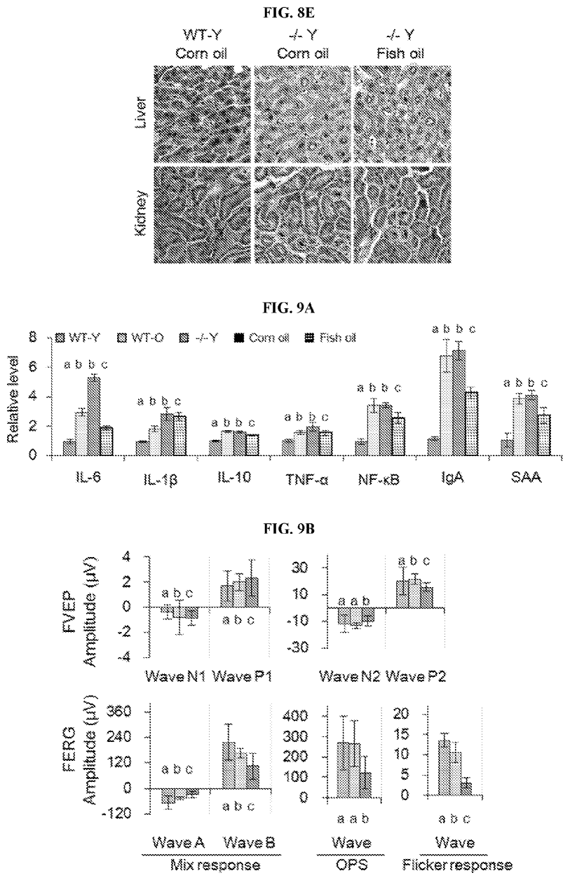

Next, how Elovl2 deficiency accelerates aging was examined. Elovl2 functions as an elongase of long fatty acids from 20: C to 28: C (FIG. 8A), and lipidomic analysis on samples from liver, brain, and plasma using Gas chromatography-mass spectrometry (GC-MS) was further performed. Saturated fatty acids (SFAs), polyunsaturated fatty acids (PUFAs) and monounsaturated fatty acids (MUFAs) were analyzed respectively. The results showed an accumulation of fatty acids with less than 20 carbons and depletion of PUFAs with carbon chains longer than 22: C, such as docosahexaenoic acid (DHA), in liver, brain and plasma of WT-O and −/− Y mice (FIG. 2E and FIG. 8B). In contrast, a depletion of PUFAs with carbon chains longer than 22: C was observed, such as DHA (FIG. 2H, and FIG. 8B). These results were consistent with reports such as from Zadravec et al. that depletion of Elovl2 impairs the lipid metabolism and activates de novo fatty acid synthesis by upregulate key transcriptional factor SREBP1 (Zadravec, et al., ELOVL2 controls the level of n−6 28:5 and 30:5 fatty acids in testis, a prerequisite for male fertility and sperm maturation in mice. J Lipid Res 52, 245-255 (2011)). Through ORO (Oil red O) staining, severe fatty acids were identified to accumulate in hepatocytes in WT-O and −/− Y mice (FIG. 2I). Using ORO (Oil red O) staining, accumulation of fatty acids was observed in hepatocytes in both WT-O and −/− Y mice (FIG. 2F). Furthermore, a steatohepatitis phenotype was detected by the ultrasonography in WT-O and −/− Y mice (FIG. 2G). In some instances, studies have shown that lipotoxicity could induce insulin resistance through the activation of stress kinases (e.g. C-jun N-terminal Kinase, i.e., JNK) and chronic ER stress (Ferre, et al., Hepatic steatosis: a role for de novo lipogenesis and the transcription factor SREBP-lc. Diabetes Obes Metab 12 Suppl 2, 83-92 (2010); Salvado, et al., Targeting endoplasmic reticulum stress in insulin resistance. Trends Endocrinol Metab 26, 438-448 (2015)). As such, glucose tolerant test (GTT) and insulin tolerant test (ITT) to test whether Elovl2 ablation could induce insulin resistance were performed. Glucose tolerance and insulin resistance phenotypes were observed in −/− Y mice in both strains (FIG. 2H and FIG. 8C).

In sum, these results showed a metabolism dysfunction in Elovl2 deficit mouse.

### PUFAs Supplement Diet is Insufficient to Fully Rescue Aging Phenotype in Elovl2 Knock Out Mice

In consideration that Elovl2 depletion diminished the synthesis of PUFAs, it was asked whether dietary supplementation of PUFAs could fully rescue the accelerated aging phenotype. In comparison to control group, dietary supplementation with PUFAs (fish oil) could slightly ameliorated the fatty acid accumulation in liver of +/−Y and −/− Y mice and improve the physiological glucose metabolic balance, but it was insufficient for complete recovery (FIGS. 2G-2H and FIG. 8C). Open field behavioral test showed that dietary supplementation with PUFAs brought slight improvement of aging phenotypes (FIG. 8D). Further, aging-related histopathological phenotypes were not relieved upon PUFAs addition (FIG. 8E). These results indicated that depletion of Elovl2 contributed to aging was not simply through the deficit of nutrient PUFAs.

Chronic Inflammation, Cellular Senescence and Adult Stem Cell Exhaustion were Induced in Elovl2 Depletion Mice

Adult stem cells play a role in maintenance of tissue function and homeostasis. They mostly reside in a metabolically inactive quiescent state but covert to a metabolically active state to restore tissue function under stress. Metabolism plays a role in maintaining an adult stem cell pool. A study by Oishi et al. showed that lipid metabolism, e.g., the PUFA synthesis, played a role in immune response (Oishi, et al., SREBP1 Contributes to Resolution of Pro-inflammatory TLR4 Signaling by Reprogramming Fatty Acid Metabolism. Cell Metab 25, 412-427 (2017)). PUFAs such as DHA and EPA regulate inflammation as the precursor of inflammation inhibitors, such as protectins, resolvins and Maresins. Considering that chronic ER stress and mitochondrial dysfunction-induced oxidative damage and lack of those pro-resolving lipid mediators after Elovl2 depletion, it was hypothesized that −/− Y mice would also experience chronic inflammation.

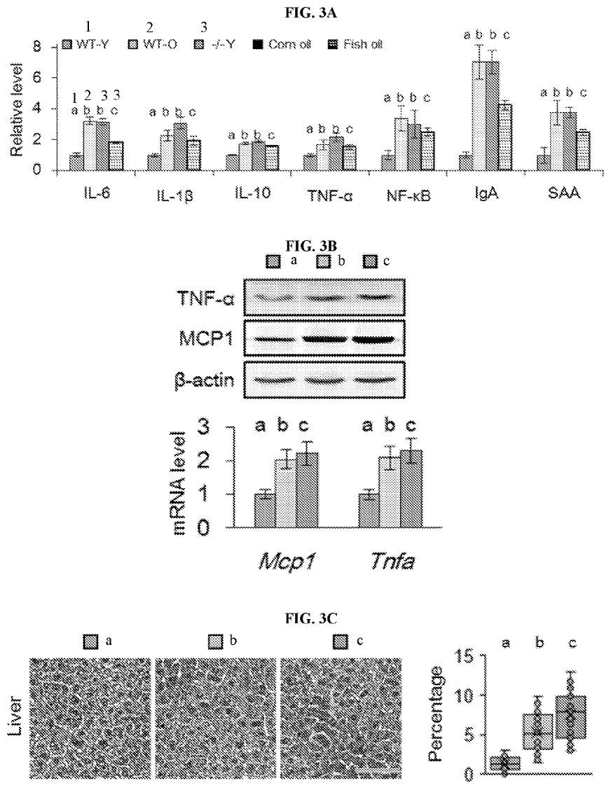

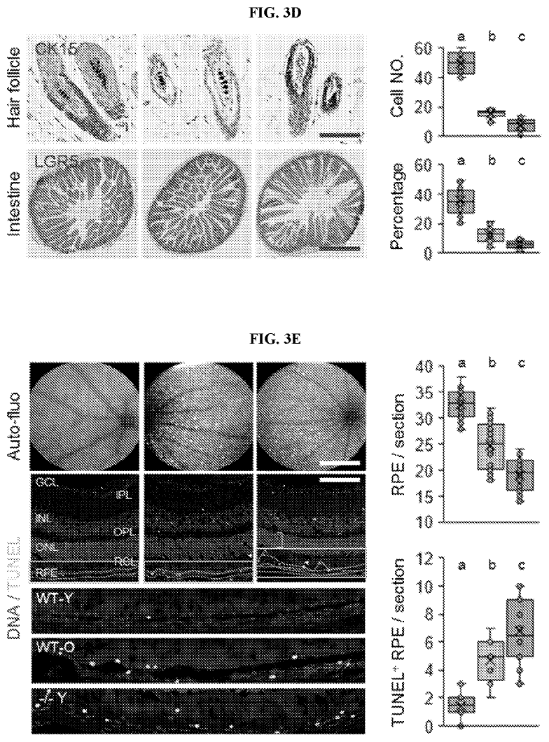

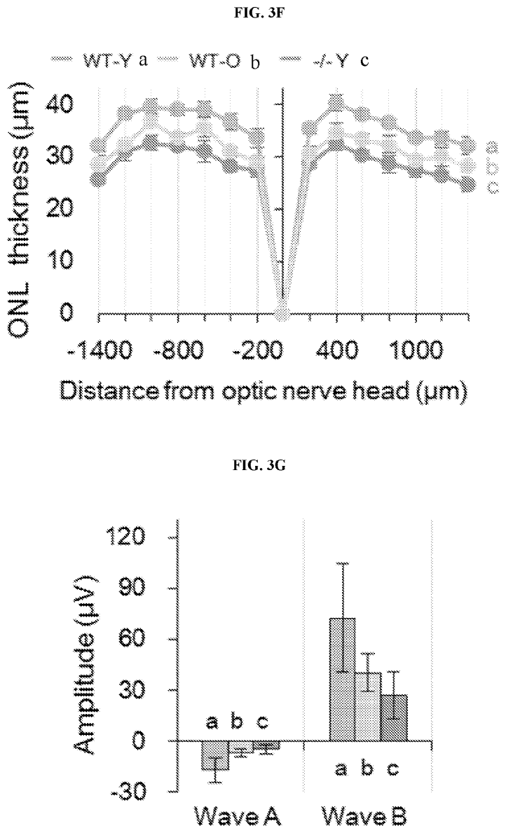

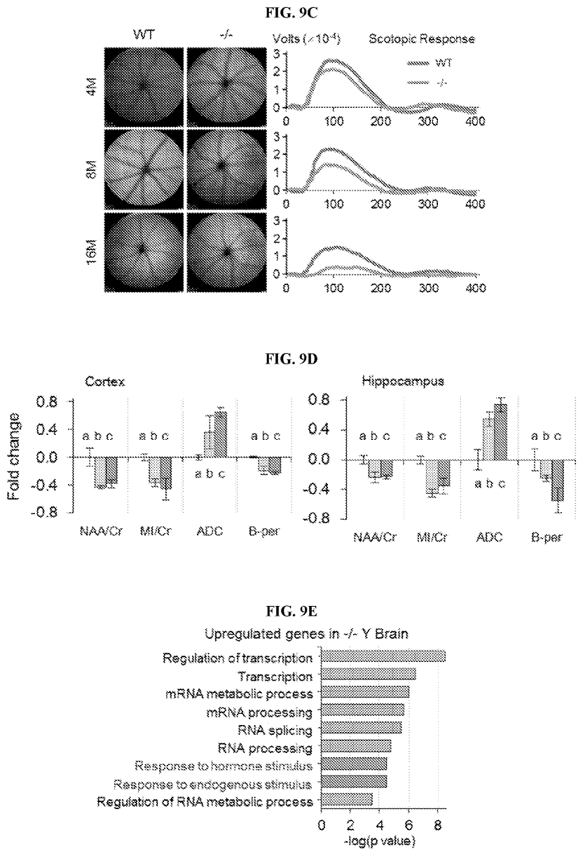

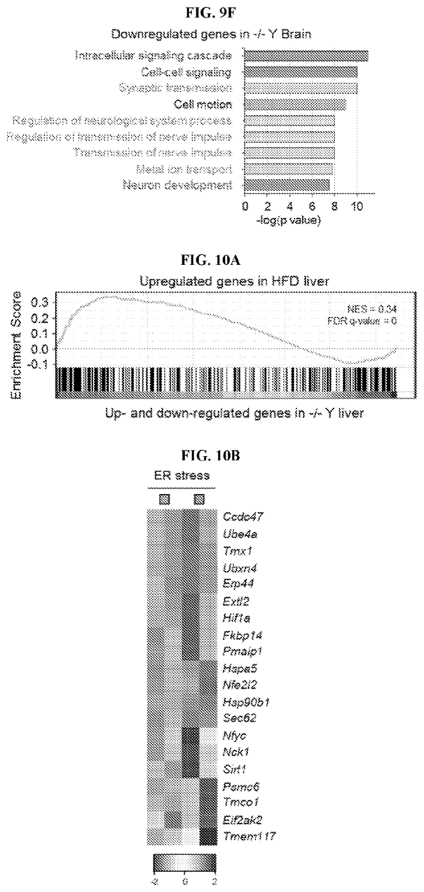

Analysis of blood samples showed an increase of inflammatory factors in −/− Y and WT-O mice (FIG. 3A, and FIG. 9A). The inflammation status in liver was also examined. An increased level of MCP1 and TNF-α were detected in WT-O and −I− Y mice (FIG. 3B). An increase of fibrosis was detected in liver, heart, and kidney (FIG. 3C) and a loss of stem cell population was detected in hair follicle (CK15) and intestine (LGR5)(FIG. 3D).

Next, it was asked whether Elovl2 ablation-caused inflammation and multiple tissue fibrosis would impair tissue and organ function. Firstly, retinal structure and ocular function were examined in these mice. Increased TUNEL staining on retina especially the RPE layer (FIG. 3E) and the decreased thickness of NRL layer (FIG. 3F) indicated the increase of senescent signals. The visual function declines of photoreceptors were found in retina of −/− Y and WT-O mice (FIG. 3G, and FIG. 9B), coincident with the appearance of drusen (FIG. 3E and FIG. 9C). Secondly, the cerebral cortex and hippocampus structure were examined by MRI and abnormity was detected in −/− Y and WT-O mice (FIG. 9D). RNA-sequence analysis also showed a remarkable abnormal expression profile and impaired functional gene expression in brain of −/−Y mice (FIGS. 9E-9F). These results indicated that failure to resolve endogenous or extrinsic inflammatory stimuli in Elovl2 depletion tissues lead to chronic inflammation, exhaustion of stem cells and loss of tissue functions which would be the driver of aging.

### Various Metabolic and Aging-Related Pathways are Affected by Elovl2 Depletion.

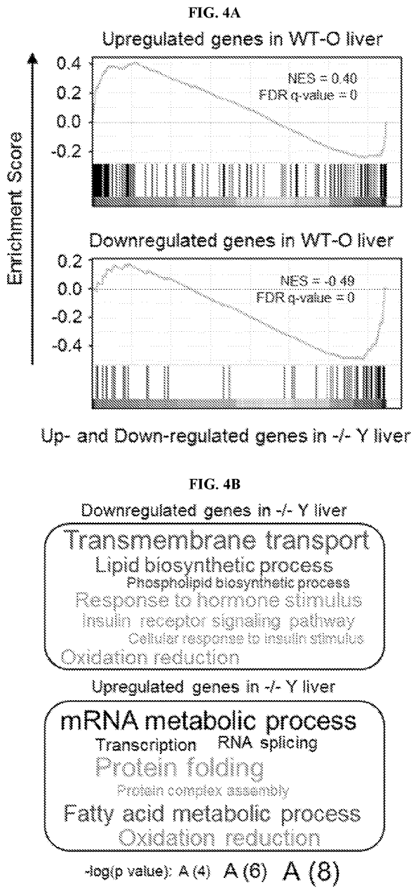

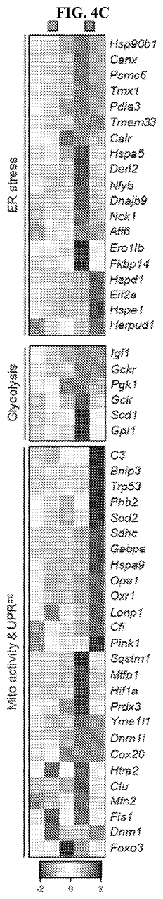

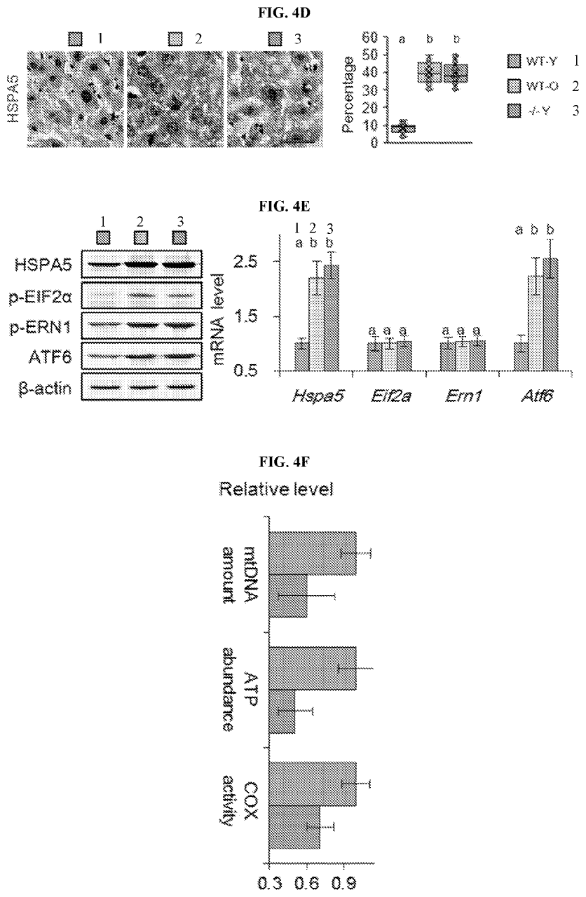

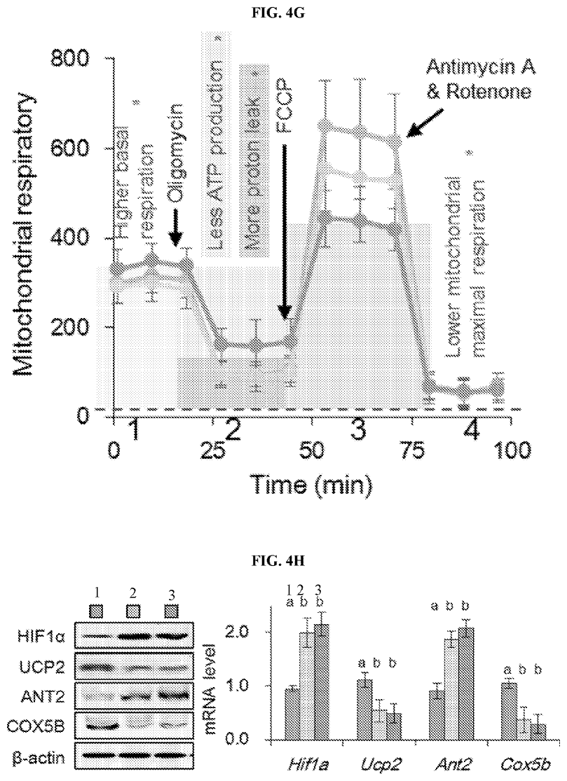

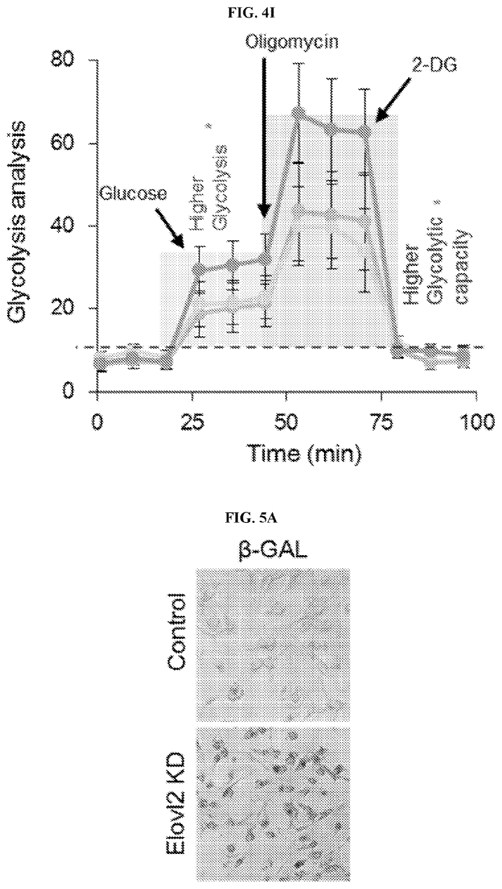

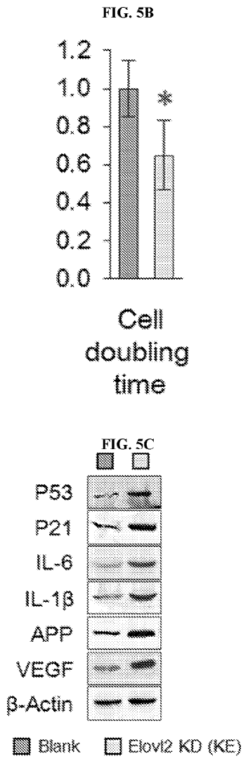

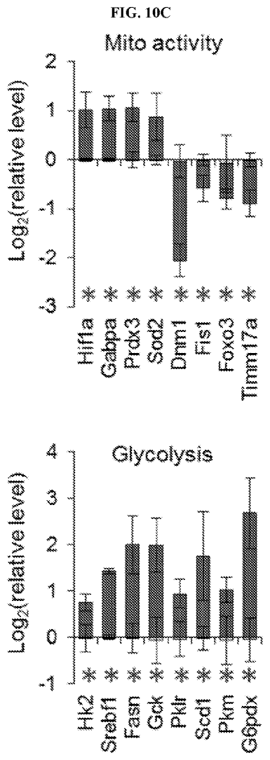

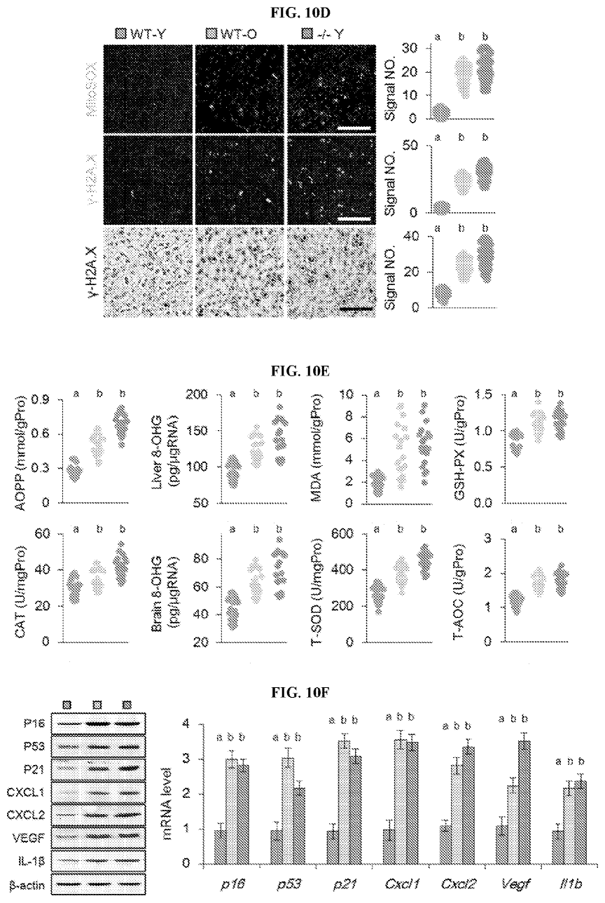

In order to reveal the mechanism of how the Elovl2 depletion accelerates aging through metabolism in molecular level, RNA-Sequence was performed on liver and brain sample from WT-Y and −/− Y mice. Compared to up-regulated genes in WT-O mice reported in White et al., the up-regulated genes identified in the current study were also enriched in the −/− Y mice (FIG. 4A)(White et al., Comprehensive transcriptional landscape of aging mouse liver. BMC Genomics 16, 899 (2015)). Further the down-regulated genes showed a consistent pattern relative to the down-regulated genes identified in White et al. (FIG. 4A). In addition, it was found that −/− Y mice have similar expression profile to HFD mice, which verified that Elovl2 ablation resulted in fatty acid accumulation (FIG. 10A). 1,867 differentially expressed genes (2-fold changes and p value <0.05) were identified in −/− Y mice liver versus WT-Y liver, with 1,084 genes up-regulated and 783 genes down-regulated. Gene Ontology (GO) enrichment analyses showed that fatty acid/lipid metabolism, cellular responses to insulin stimulus, mitochondrial function and uncoupled protein response were mis-regulated (FIG. 4B). Notably, it was found that the ER stress associated genes were up-regulated (FIG. 4C and FIG. 10B). Further, mitochondrial function-associated genes, such as genes involved in fatty acid β-oxidation process and insulin receptor signaling pathway, were down-regulated, mitochondrial uncoupled protein response (UPR^{mt})— and glycolysis-associated genes were up-regulated (FIG. 4C). Consistent with RNA-Seq data, RT-qPCR also showed the same expression patterns (FIG. 10C).

### Lipid Metabolic Disorder Causes ER Stress Response and Mitochondrial Dysfunction

Mitochondria participates aging process in a wide range of aspects, such as energy production, ROS generation and mitochondrial uncoupled protein response (Mito-UPR). Also, mitochondria contribute to the shifting of cellular senescence state. Based in part on the RNA-sequence data analysis, it was hypothesized that the Elovl2 depletion disturbed lipid metabolism and up-regulated fatty acid biosynthesis, followed by ER stress induced by the fatty acid precursor accumulation on ER, and thus mitochondrial dysfunction, which subsequently promoted aging. To verify the hypothesis, ER stress and mitochondrial function in liver tissues were measured. ER stress markers such as HSPA5, phosphorylated EIF2α (p-EIF2α), p-ERN1, and ATF6 were up-regulated in WT-O and −/− Y mice (FIG. 4D-FIG. 4E). Markers such as mtDNA content, ATP abundance, and COX activity were decreased in WT-O and −/− Y mice while the WT-Y and +/−Y mice showed a relatively high level of mitochondrial activity (FIG. 4F). The Agilent Seahorse XF Cell Mito Stress Test was used to test the key parameters of mitochondrial function by direct measuring the oxygen consumption rate (OCR) of hepatocytes from WT-O and −/− Y mice. For basal respiration, the oxygen consumption rate was found to increase in −/− Y mice (FIG. 4G, 1). The increase was not abrogated by oligomycin treatment, which suggested the increased uncoupled respiration and proton leak as well as the mitochondrion damage (FIG. 4G, 2). It was found that the expression of HiF1α and ANT2 were up-regulated, while the expression of UCP2 was down-regulated (FIG. 4H), indicating there is hypoxia-mediated mitochondrial dysfunction in −/− Y mice which may also result from superfluous fatty acids (Lee, et al., Increased Adipocyte 02 Consumption Triggers HIF-1α, Causing Inflammation and Insulin Resistance in Obesity. Cell 157, 1339-1352 (2014); Hetz, et al., The unfolded protein response: controlling cell fate decisions under ER stress and beyond. Nat Rev Mol Cell Biol 13, 89-102 (2012)).

After FCCP addition, the results showed a lower mitochondrial maximal respiration of −/− Y mice (FIG. 4G, 3), consistent with the lower level of COX5B, which reflected the lower activity of oxidation respiratory chain (FIG. 4H). In addition, it was found that −/− Y mice showed an increased glycolysis activity (FIG. 4I). A switch of metabolism from oxidative phosphorylation to glycolysis which was known as Warburg in both cancerous and senescent cells was observed, which was also found from the RNA-seq data (FIG. 4C).

These results demonstrated the occurrence of mitochondrial damage and dysfunction where superfluous fatty acids induced hypoxia and high oxygen consumption were involved. One of the consequences of chronic ER stress and mitochondrial dysfunction is the oxidative damage on cellular level. Via different detection means, it was found that oxidative damages happened in both mitochondrion (MitoSOX for mitochondrial superoxide, FIG. 10D) and nuclei (γ-H2AX for DNA oxidative damage, FIG. 10D). Oxidative damages were also observed on various levels including protein (FIG. 10E, AOPP), lipid (FIG. 10E, MDA), and RNA (FIG. 10E, 8-OHG). In addition, antioxidative enzymes were also overactivated (FIG. 10E, GSH-PX, CAT, T-SOD, and TAC).

Upon such severe oxidative damage, higher cellular senescent markers were detected by western blot and RT-qPCR in −/− Y and WT-O mice (FIG. 10F).

### Elovl2 Deficiency in Human RPE Cells Induces an AMD Phenotype.

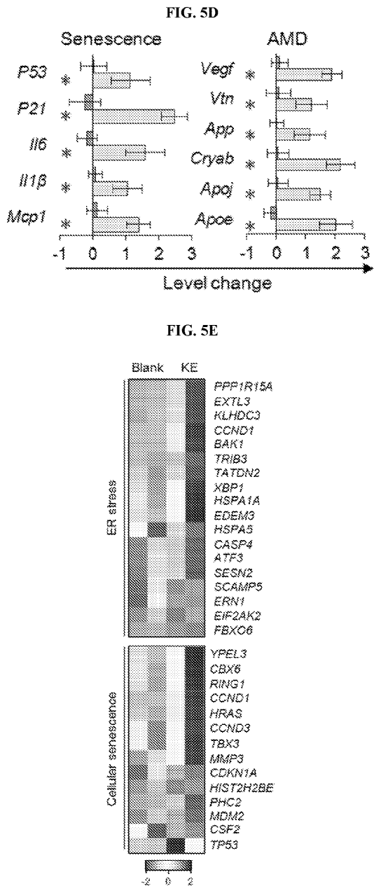

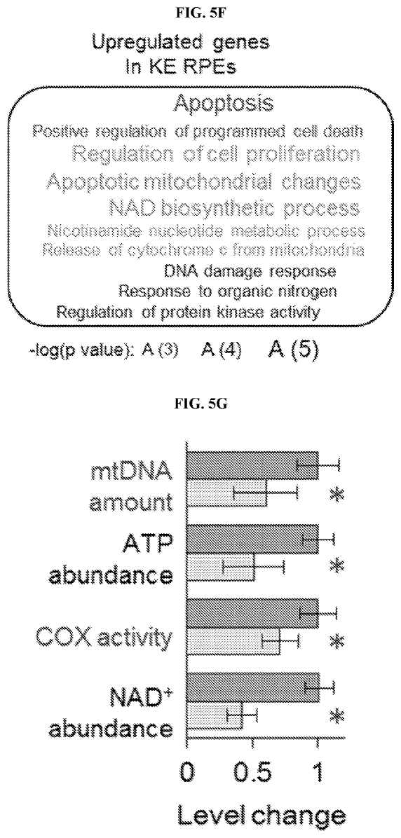

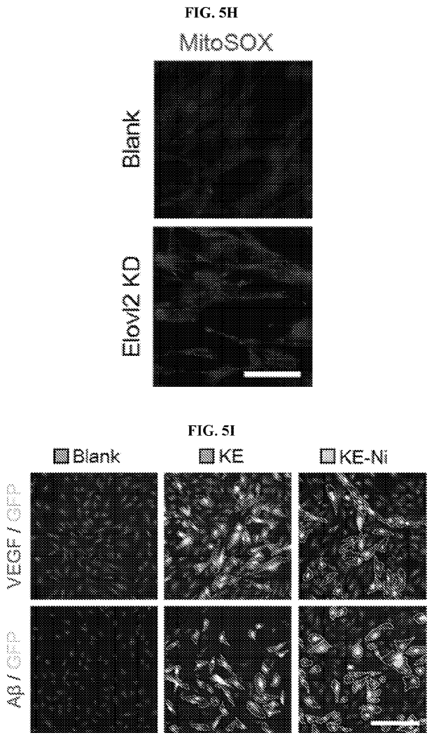

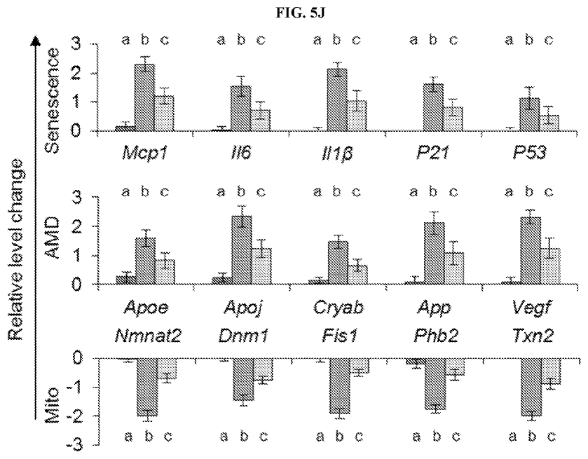

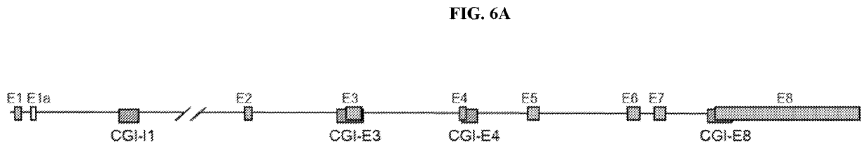

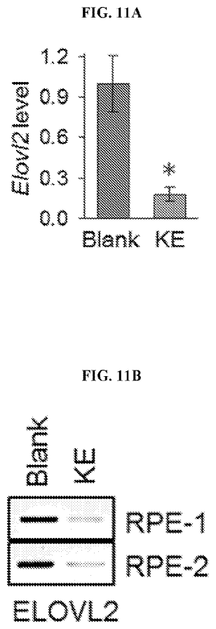

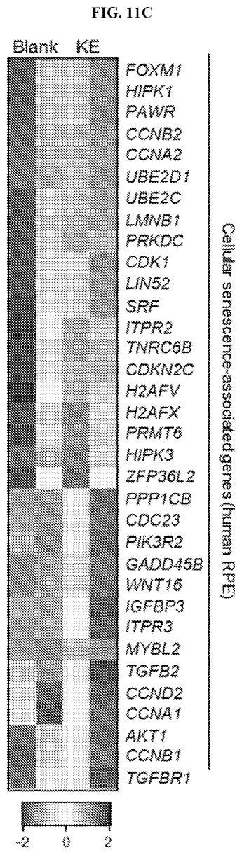

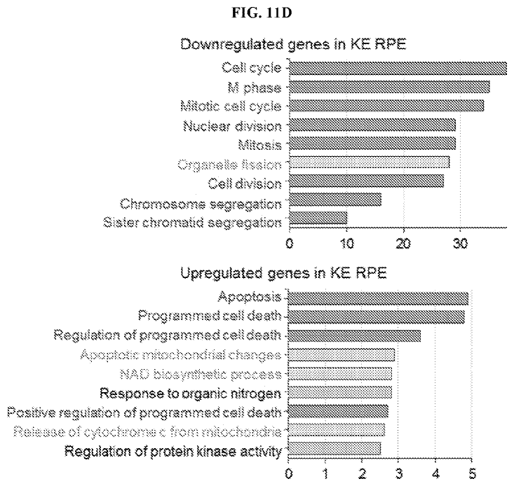

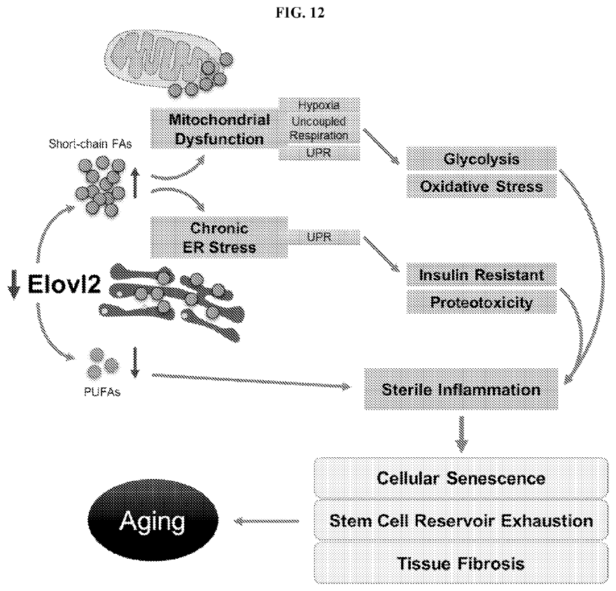

Since a wide range of metabolic changes were found related to AMD phenotype, whether Elovl2 ablation could result in AMD phenotype in human was further examined. Elovl2 knockdown RPE cell lines (KE) was developed via lentiviral delivery of shRNAs to human primary RPE cells (generated from healthy donors)(FIG. 11A and FIG. 11B). The deletion of Elovl2 resulted in cellular senescence (FIG. 5A) and impaired proliferation (FIG. 5B). Upregulation of senescence-associated secretary phenotype (SASP) markers including P53, P21, IL-lb, IL-6 and MCP1 in KE cells was also detected (FIG. 5C). Additional senescence and AMD markers were also found to increase in RNA and protein level in KE cells (FIG. 5D). These results indicated that an AMD model with increased ASAP phenotype can be generated by Elovl2 depletion in human RPE cells.

Elovl2 depletion led to fatty acid accumulation that triggered chronic ER stress and mitochondrial dysfunction (FIG. 4). Thus, it was hypothesized that these impairments also took parts in the Elovl2 deficiency-induced human AMD model. Indeed, from the RNA-sequence analysis, an increase of chronic ER stress besides cellular senescence was detected in KE cells (FIG. 5E and FIG. 11C). Moreover, the GO term analysis showed mitochondrial dysfunction in KE cells, with dysregulation of genes associated with mitochondrial function, such as “NAD biosynthetic process” and “apoptotic mitochondria' changes” (FIG. 5F and FIG. 11D). In addition, it was found that the KE cells showed a decreased amount of mitochondrion and loss of mitochondrial function with decline of the mtDNA content, ATP abundance, COX activity, and NAD+ abundance (FIG. 5G). Furthermore, an accumulation of oxidative damages was detected in mitochondria (FIG. 5H), which reflected severe oxidative damage in KE cells similar to cells of −/− Y mice. Above all, Elovl2 deficiency led to an increase of oxidative damages caused by chronic ER stress and mitochondrial dysfunction both in mouse model and human cell model.

Further, it was test whether the mitochondria activator nicotinamide (Ni) could rescue the AMD phenotype in Elovl2 knock-down cells. Treatment with Ni during the culturing of KE cells reversed the abnormal expression of cellular senescence genes and reduced mitochondrial dysfunction, thus causing a reduction of AMID markers (FIGS. 5I-5J). These results indicated that restoration of mitochondrial activity and clearance of ER stress can ameliorate AMD phenotype caused by Elovl2 depletion.

### Example 2

### In Vivo Administration of an Exemplary AAV-Based ELOVL2 Construct

An exemplary AAV-based ELOVL2 vector is administered intravenously into a group of 20 Elovl2 knock out mice with each mouse receiving about 1×10¹²AAV vector particles. At a respective time point post-injection, plasma or tissue samples are obtained and processed and ELOVL2 expression are subsequently determined.

FIG. 14 illustrates an exemplary AAV-based ELOVL2 construct described herein.

### Example 3

TABLE 1
illustrates exemplary protein sequences of
ELOVL2 and KLF14 described herein.

| | | SEQ |
|---|---|---|
| | | ID |
| Protein | Sequence | NO: |
| ELOVL2 | MEHLKAFDDEINAFLDNMFGPRDSRVRG | 1 |
| (NCBI Ref No.: | WFMLDSYLPTFFLTVMYLLSIWLGNKYM | |
| NP_060240.3) | KNRPALSLRGILTLYNLGITLLSAYMLA | |
| | ELILSTWEGGYNLQCQDLTSAGEADIRV | |
| | AKVLWWYYFSKSVEFLDTIFFVLRKKTS | |
| | QITFLHVYHHASMFNIWWCVLNWIPCGQ | |
| | SFFGPTLNSFIHILMYSYYGLSVFPSMH | |
| | KYLWWKKYLTQAQLVQFVLTITHTMSAV | |
| | VKPCGFPFGCLIFQSSYMLTLVILFLNF | |
| | YVQTYRKKPMKKDMQEPPAGKEVKNGFS | |
| | KAYFTAANGVMNKKAQ | |
| KLF14 | MSAAVACLDYFAAECLVSMSAGAVVHRR | 2 |
| (NCBI Ref No.: | PPDPEGAGGAAGSEVGAAPPESALPGPG | |
| | PPGPASVPQLPQVPAPSPGAGGAAPHLL | |
| | AASVWADLRGSSGEGSWENSGEAPRASS | |
| | GFSDPIPCSVQTPCSELAPASGAAAVCA | |
| NP_619638.2) | PESSSDAPAVPSAPAAPGAPAASGGFSG | |
| | GALGAGPAPAADQAPRRRSVTPAAKRHQ | |
| | CPFPGCTKAYYKSSHLKSHQRTHTGERP | |
| | FSCDWLDCDKKFTRSDELARHYRTHTGE | |
| | KRFSCPLCPKQFSRSDHLTKHARRHPTY | |
| | HPDMIEYRGRRRTPRIDPPLTSEVESSA | |
| | SGSGPGPAPSFTTCL | |

Embodiment 1: A method of treating a subject in need thereof, comprising: administering to the subject a composition comprising an active agent that up-regulates ELOVL fatty acid elongase 2 (ELOVL2) expression and a pharmaceutically acceptable carrier.

Embodiment 2: The method of embodiment 1, wherein the active agent comprises a vector comprising a polynucleotide encoding ELOVL2 or a functionally-active fragment thereof.

Embodiment 3: The method of embodiment 2, wherein the polynucleotide encodes a polypeptide comprising at least 80%, 85%, 90%, 95%, or 99% sequence identity to SEQ ID NO: 1.

Embodiment 4: The method of embodiment 2 or 3, wherein the vector comprises a viral vector.

Embodiment 5: The method of embodiment 4, wherein the viral vector comprises an adeno-associated virus (AAV)-based vector.

Embodiment 6: The method of embodiment 5, wherein the AAV-based vector comprises AAV-based vector of serotype 1 (AAV1), AAV-based vector of serotype 2 (AAV2), AAV-based vector of serotype 3 (AAV3), AAV-based vector of serotype 4 (AAV4), AAV-based vector of serotype 5 (AAVS), AAV-based vector of serotype 6 (AAV6), AAV-based vector of serotype 7 (AAV7), AAV-based vector of serotype 8 (AAV8), AAV-based vector of serotype 9 (AAV9), or a humanized AAV-based vector.

Embodiment 7: The method of embodiment 4, wherein the viral vector comprises an adenovirus-based vector, an alphavirus-based vector, a herpesvirus-based vector, a retrovirus-based vector, a lentivirus-based vector, or a vaccinia virus-based vector.

Embodiment 8: The method of any one of the embodiments 2-7, wherein the vector comprises a cell or tissue-specific promoter.

Embodiment 9: The method of embodiment 8, wherein the cell or tissue-specific promoter is an endogenous promotor specific to the cell type of interest.

Embodiment 10: The method of embodiment 8, wherein the cell or tissue-specific promoter is an exogenous promotor specific to the cell type of interest.

Embodiment 11: The method of any one of the embodiments 2-10, wherein the vector comprises a microbial promoter.

Embodiment 12: The method of embodiment 11, wherein the microbial promoter comprises SV40 or cytomegalovirus (CMV) immediate-early promoter.

Embodiment 13: The method of any one of the embodiments 2-12, wherein the vector comprises an enhancer, an inverted terminal repeats (ITR), a capsid, polyadenylation signal, a signal sequence, or a combination thereof.

Embodiment 14: The method of any one of the embodiments 2-13, wherein the vector comprises a selectable marker.

Embodiment 15: The method of embodiment 14, wherein the selectable marker comprises a polynucleotide encoding a fluorescent protein.

Embodiment 16: The method of embodiment 15, wherein the fluorescent protein comprises green fluorescent protein (GFP), enhanced green fluorescent protein (EGFP), Superfolder GFP, enhanced cyan fluorescent protein (ECFP), DsRed fluorescent protein (DsRed2FP), mTurquoise, mVenus, Emerald, Azami Green, mWasabi, TagFGP, TurboFGP, AcGFP, ZsGreen, T-Sapphire, enhanced blue fluorescent protein (EBFP), Azurite, mTagBFP, Cerulean, CyPet, AmCyanl, Midori-Ishi Cyan, TagCFP, mTFP1, enhanced yellow fluorescent protein (EYFP), Topaz, MCitrine, YPet, TagYFP, PhiYFP, ZsYellow 1, mBanana, Kusabira Orange, Kusabira Orange2, mOrange, dTomato, TagRFP, TagRFP-T, DsRed, DsRed-Express (T1), mTangerine, mRuby, mApple, mStrawberry, AsRed2, mRFP1, JRed, mCherry, HcRedl, mRaspberry, dKeima-Tandem, mPlum, or AQ143.

Embodiment 17: The method of any one of the embodiments 2-16, wherein the vector comprises a polynucleotide encoding an elongation factor 1-alpha (EF1α).

Embodiment 18: The method of any one of the embodiments 2-17, wherein the vector comprises a polynucleotide encoding a Klarsicht, ANC-1, Syne Homology (KASH) domain.

Embodiment 19: The method of embodiment 1, wherein the active agent inhibits activation of chromodomain-helicase-DNA-binding protein 4 (CHD4).

Embodiment 20: The method of any one of the embodiments 1-19, wherein the composition is administered systemically.

Embodiment 21: The method of any one of the embodiments 1-19, wherein the composition is administered as a local injection.

Embodiment 22: The method of any one of the embodiments 1-21, wherein the composition is formulated for parenteral administration.

Embodiment 23: The method of any one of the embodiments 1-19, wherein the composition is formulated for oral or intranasal administration.

Embodiment 24: The method of any one of the embodiments 1-23, wherein a reduced ELOVL2 expression level correlates with an increase in accumulation of a plurality of fatty acids with less than 22 carbon chains.

Embodiment 25: The method of embodiment 24, wherein the plurality of fatty acids comprises saturated fatty acids, monounsaturated fatty acids, or a combination thereof.

Embodiment 26: The method of any one of the embodiments 1-25, wherein an elevated expression of ELOVL2 reduces or slows-down an aging phenotype.

Embodiment 27: The method of embodiment 26, wherein the aging phenotype comprises hair loss, a decrease in bone density, a decrease in endurance, a decrease in muscle strength, or neurodegeneration.

Embodiment 28: The method of any one of the embodiments 1-25, wherein an elevated expression of ELOVL2 treats age-related macular degeneration (AMD).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of reducing or slowing down an aging phenotype in a subject in need thereof, comprising administering to the subject a composition comprising a therapeutic agent that reduces or slows down the aging phenotype,
wherein the therapeutic agent comprises a vector comprising a polynucleotide encoding ELOVL2 or a functionally active fragment thereof, and
wherein the vector is a viral vector selected from the group consisting of an alphavirus-based vector, a herpesvirus-based vector, a retrovirus-based vector, a lentivirus-based vector, and a vaccinia virus-based vector.

2. The method of claim 1, wherein the vector is an alphavirus-based vector.

3. The method of claim 1, wherein the vector is a herpesvirus-based vector.

4. The method of claim 1, wherein the vector is a retrovirus-based vector.

5. The method of claim 1, wherein the vector is a lentivirus-based vector.

6. The method of claim 1, wherein the vector is a vaccinia virus-based vector.

7. The method of claim 1, wherein the polynucleotide encodes a polypeptide comprising at least 80%, 85%, 90%, 95%, or 99% sequence identity to SEQ ID NO: 1.

8. The method of claim 1, wherein the vector comprises a promoter, an enhancer, an inverted terminal repeat (ITR), a capsid, polyadenylation signal, a signal sequence, or a combination thereof.

9. The method of claim 1, wherein the composition further comprises a pharmaceutically acceptable carrier.

10. The method of claim 1, wherein the aging phenotype comprises hair loss.

11. The method of claim 1, wherein the aging phenotype comprises a decrease in bone density.

12. The method of claim 1, wherein the aging phenotype comprises a decrease in endurance.

13. The method of claim 1, wherein the aging phenotype comprises a decrease in muscle strength.

14. The method of claim 1, wherein the aging phenotype comprises neurodegeneration.

15. The method of claim 1, wherein the aging phenotype comprises age-related macular degeneration.

16. The method of claim 1, wherein the subject is human.


SEQUENCE LISTING (6 sequences)

SEQ ID NO: 1 - protein, 296 aa, Homo sapiens
MEHLKAFDDEINAFLDNMFGPRDSRVRGWFMLDSYLPTFFLTVMYLLSIWLGNKYMKNRPALSLRGILTLYNLGITLLSAYMLAELILSTWEGGYNLQCQDLTSAGEADIRVAKVLWWYYFSKSVEFLDTIFFVLRKKTSQITFLHVYHHASMFNIWWCVLNWIPCGQSFFGPTLNSFIHILMYSYYGLSVFPSMHKYLWWKKYLTQAQLVQFVLTITHTMSAVVKPCGFPFGCLIFQSSYMLTLVILFLNFYVQTYRKKPMKKDMQEPPAGKEVKNGFSKAYFTAANGVMNKKAQ

SEQ ID NO: 2 - protein, 323 aa, Homo sapiens
MSAAVACLDYFAAECLVSMSAGAVVHRRPPDPEGAGGAAGSEVGAAPPESALPGPGPPGPASVPQLPQVPAPSPGAGGAAPHLLAASVWADLRGSSGEGSWENSGEAPRASSGFSDPIPCSVQTPCSELAPASGAAAVCAPESSSDAPAVPSAPAAPGAPAASGGFSGGALGAGPAPAADQAPRRRSVTPAAKRHQCPFPGCTKAYYKSSHLKSHQRTHTGERPFSCDWLDCDKKFTRSDELARHYRTHTGEKRFSCPLCPKQFSRSDHLTKHARRHPTYHPDMIEYRGRRRTPRIDPPLTSEVESSASGSGPGPAPSFTTCL

SEQ ID NO: 3 - DNA, 21 bp, Homo sapiens
  misc_feature 1..21: note = expresssing shRNA
  source 1..21: mol_type = unassigned DNA
tggtggtactatttctccaaa

SEQ ID NO: 4 - DNA, 21 bp, Homo sapiens
  misc_feature 1..21: note = expressing sgRNA-1
  source 1..21: mol_type = unassigned DNA
tttacagattctcgagttcgc

SEQ ID NO: 5 - DNA, 20 bp, Homo sapiens
  misc_feature 1..20: note = expressing sgRNA-2
  source 1..20: mol_type = unassigned DNA
ctcgatatggctgggtaaca

SEQ ID NO: 6 - DNA, 41 bp, Homo sapiens
  misc_feature 1..41: note = expressing sgRNA-1
  source 1..41: mol_type = unassigned DNA
tttacagattctcgagttcgcctcgatatggctgggtaaca